(19) United States
(12) Patent Application Publication
(10) Pub. No.: US 2023/0293430 A1
(43) Pub. Date: Sep. 21, 2023

(54) FORMULATIONS INCLUDING 5-[3-(3-HYDROXYPHENOXY)AZETIDIN-1-YL]-5-METHYL-2,2-DIPHENYLHEXANAMIDE

(71) Applicant: Mylan Pharma UK Limited, Hertfordshire (GB)

(72) Inventors: Jane Burrows, Kent (GB); Gavin Magee, Kent (GB); Claire Marcellin, Kent (GB)

(73) Assignee: Mylan Pharma UK Limited, Hertfordshire (GB)

(21) Appl. No.: 18/012,110

(22) PCT Filed: Jun. 25, 2021
(86) PCT No.: PCT/IB2021/000496

§ 371 Date: Dec. 21, 2022

Related U.S. Application Data
(60) Provisional application No. 63/044,404, filed on Jun. 26, 2020.

(51) Int. Cl. A61K 9/00; A61K 31/397; A61M 15/00; A61K 47/26; A61K 31/137; A61K 31/567

(52) U.S. Cl. CPC A61K 9/0075; A61K 31/397; A61M 15/0003; A61K 47/26; A61K 31/137; A61K 31/567; A61M 2202/064

(57) ABSTRACT

Powder formulations for use in dry powder inhalers and methods of making them are provided. A powder formulation can include from about 0.01% to about 90% by weight of a carboxamide compound comprising 5-[3-(3-Hydroxyphenoxy)azetidin-1-yl]-5-methyl-2,2-diphenyl-hexanamide or a pharmaceutically acceptable salt thereof and an excipient. Another powder formulation includes in addition to the carboxamide compound or its hydrochloride salt a long acting β2 adrenoreceptor agonist and an inhalable corticosteroid.

### CROSS REFERENCE TO RELATED APPLICATION(S)

This application claims priority to U.S. Provisional Application Ser. No. 63/044,404, filed on Jun. 26, 2020, the entire disclosure of which is hereby incorporated by reference in its entirety into the present disclosure.

### BACKGROUND

### Field

Cholinergic muscarinic receptors are members of the G-protein coupled receptor super-family and are further divided into 5 subtypes, M₁ to M₅. Muscarinic receptor sub-types are widely and differentially expressed in the body. Genes have been cloned for all 5 sub-types and of these, M₁, M₂ and M₃ receptors have been extensively pharmacologically characterized in animal and human tissue. M₁ receptors are expressed in the brain (cortex and hippocampus), glands and in the ganglia of sympathetic and parasympathetic nerves. M₂ receptors are expressed in the heart, hindbrain, smooth muscle and in the synapses of the autonomic nervous system. M₃ receptors are expressed in the brain, glands and smooth muscle. In the airways, stimulation of M₃ receptors evokes contraction of airway smooth muscle leading to bronchoconstriction, while in the salivary gland, M₃ receptor stimulation increases fluid and mucus secretion leading to increased salivation. M₂ receptors expressed on smooth muscle are understood to be pro-contractile while pre-synaptic M₂ receptors modulate acetylcholine release from parasympathetic nerves. Stimulation of M₂ receptors expressed in the heart produces bradycardia.

Short and long-acting muscarinic antagonists are used in the management of asthma and COPD; these include the short acting agents Atrovent® (ipratropium bromide) and Oxivent® (oxitropium bromide) and the long acting agent Spiriva® (tiotropium bromide). These compounds produce bronchodilation following inhaled administration. In addition to improvements in spirometric values, anti-muscarinic use in chronic obstructive pulmonary disease (COPD) is associated with improvements in health status and quality of life scores.

Chronic obstructive pulmonary disease (COPD) is a multi-component disease characterized by airflow limitation and airway inflammation. Exacerbations of COPD have a considerable impact on the quality of life, daily activities and general well-being of patients and are a great burden on the health system. Thus, the aims of COPD management include not only relieving symptoms and preventing disease progression, but also preventing and treating exacerbations.

While available therapies improve clinical symptoms and decrease airway inflammation, they do not unequivocally slow long-term progression or address all disease components. With the burden of COPD continuing to increase, research into new and improved treatment strategies to optimize pharmacotherapy is ongoing, and in particular, combination therapies, with a view to their complementary modes of action enabling multiple components of the disease to be addressed.

Bronchodilators such as β2-agonists and anticholinergics are the mainstay of symptom management in mild and moderate disease, prescribed on an as-needed basis for mild COPD and as a maintenance therapy for moderate COPD. These bronchodilators are efficiently administered by inhalation, thus increasing the therapeutic index and reducing side effects of the active material.

For the treatment of more severe COPD, guidelines recommend the addition of inhaled corticosteroids (ICSs) to long-acting bronchodilator therapy. Combination therapies have been investigated with respect to their complementary modes of action enabling multiple components of the disease to be addressed. Triple therapy, combining an anticholinergic with an ICS and a long-acting β2-agonist (LABA), may provide clinical benefits additional to those associated with each treatment alone in patients with moderate to severe COPD.

Often combination formulations are used in pressurized metered dose inhalers (pMDIs). However, pMDI formulations may have some disadvantages, in particular in elderly patients, mostly due to their difficulty to synchronize actuation from the device with inspiration. Dry powder inhalers (DPIs) can be an alternative to pMDIs for the administration of drugs to airways and lung.

Therefore, it would be beneficial to provide a powder formulation for administration with DPIs comprising an anticholinergic and optionally an inhaled corticosteroid, and optionally a long-acting β2-agonist as active ingredients. Methods of making the powder formulation for administration with DPIs comprising an anticholinergic and optionally an inhaled corticosteroid, and optionally a long-acting β2-agonist as active ingredients would also be beneficial.

### SUMMARY

In some non-limiting embodiments, provided is a powder formulation for use in a dry powder inhaler is provided for monotherapy or prophylaxis of numerous disorders in which muscarinic receptors are involved or in which antagonism of this receptor may induce a benefit for allergic and non-allergic airways diseases (e.g., asthma, COPD). Methods of making the powder formulation are also provided for administration with DPIs comprising an anticholinergic and optionally an inhaled corticosteroid, and optionally a long-acting β2-agonist.

In some non-limiting embodiments, the powder formulation comprises, consists essentially of, or consists of from about 0.01% by weight to about 90% by weight of a carboxamide compound of formula I

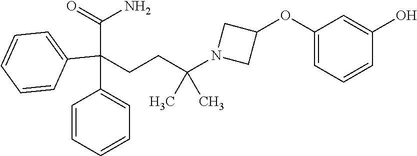 [SMILES: CC(C)(CCC(C(N)=O)(c1ccccc1)c1ccccc1)N1CC(Oc2cccc(O)c2)C1]

or a pharmaceutically acceptable salt thereof and an excipient. In other aspects, the carboxamide compound of the powder formulation is a pharmaceutically acceptable salt, specifically, a carboxamide hydrochloride of formula II

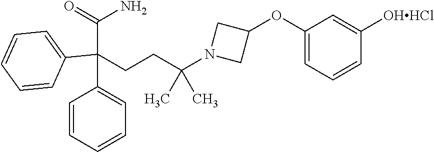

also known as 5-[3-(3-Hydroxyphenoxy)azetidin-1-yl]-5-methyl-2,2-diphenylhexanamide hydrochloride.

In other non-limiting embodiments, the powder formulation is a combination of an anticholinergic or long-acting muscarinic antagonist (LAMA), for example, a carboxamide compound of formula I or formula II, a long acting (32 adrenoceptor agonist (LABA), an inhaled corticosteroid (ICS) and an excipient to provide a triple therapy or prophylaxis of disorders in which such treatment would be beneficial. In certain aspects, the long-acting muscarinic antagonist comprises, consists essentially of, or consists of the carboxamide compound of formula I

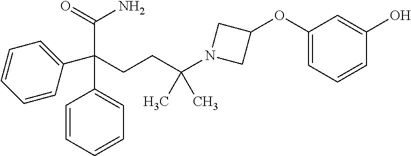 [SMILES: CC(C)(CCC(C(N)=O)(c1ccccc1)c1ccccc1)N1CC(Oc2cccc(O)c2)C1]

or a pharmaceutically acceptable salt thereof, the long acting (32 adrenoceptor agonist comprises, consists essentially of, or consists of salmeterol xinafoate and the corticosteroid comprises, consists essentially of, or consists of fluticasone propionate. The carboxamide compound of formula I is also known as 5-[3-(3-Hydroxyphenoxy)azetidin-1-yl]-5-methyl-2,2-diphenyl-hexanamide.

In some non-limiting embodiments, provided is a method of making a powder formulation for use in a dry powder inhaler, the method comprising mixing a powder formulation comprising from about 0.01% by weight to about 90% by weight of a carboxamide compound of formula I

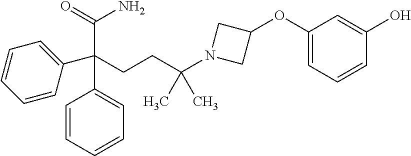 [SMILES: CC(C)(CCC(C(N)=O)(c1ccccc1)c1ccccc1)N1CC(Oc2cccc(O)c2)C1]

or a pharmaceutically acceptable salt thereof with an excipient.

In some non-limiting embodiments, provided is a method of making a powder formulation for use in a dry powder inhaler, the method comprising mixing a powder comprising a carboxamide compound of formula I

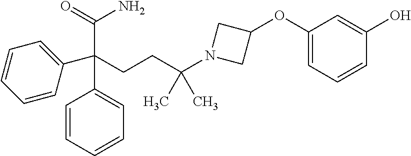 [SMILES: CC(C)(CCC(C(N)=O)(c1ccccc1)c1ccccc1)N1CC(Oc2cccc(O)c2)C1]

or a pharmaceutically acceptable salt thereof, with a pharmaceutically acceptable excipient to form a mixture and adding a long acting β2 adrenoceptor agonist or a corticosteroid or both to the mixture.

Various aspects of the present disclosure may be further characterized by one or more of the following clauses:
Clause 1: 1. A powder formulation for use in a dry powder inhaler, the powder formulation comprising from about 0.01% by weight to about 90% by weight of a carboxamide compound of formula I

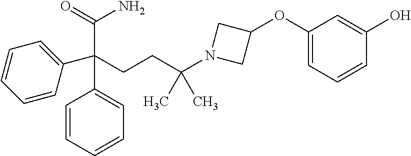 [SMILES: CC(C)(CCC(C(N)=O)(c1ccccc1)c1ccccc1)N1CC(Oc2cccc(O)c2)C1]

or a pharmaceutically acceptable salt thereof and an excipient.
Clause 2: The powder according to clause 1, wherein the carboxamide compound is a compound of formula II

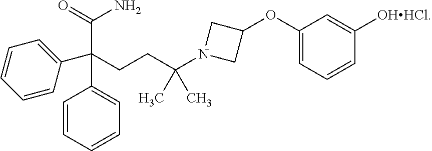

Clause 3: The powder according to clause 1 or clause 2, wherein the carboxamide compound is 5-[3-(3-Hydroxyphenoxy)azetidin-1-yl]-5-methyl-2,2-diphenylhexanamide hydrochloride.
Clause 4: The powder according to any one of clauses 1-3, wherein the powder is dry and comprises: (i) from about 0.25% by weight to about 10% by weight of the carboxamide compound of formula I; or (ii) from about 0.34% by weight to about 6.00% by weight of the carboxamide compound of formula I.
Clause 5: The powder according to any one of clauses 1-4, wherein the inhaler comprises a plurality of pockets, each pocket configured to contain the carboxamide compound at a nominal fill weight of from about 6.3 mg to about 7.0 mg.
Clause 6: The powder according to any one of clauses 1-5, wherein a nominal dose of carboxamide compound of formula I

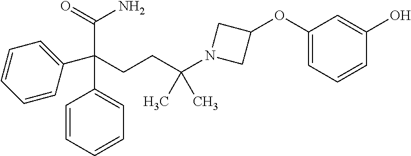 [SMILES: CC(C)(CCC(C(N)=O)(c1ccccc1)c1ccccc1)N1CC(Oc2cccc(O)c2)C1]

per nominal pocket fill comprises: (i) from about 0.018 mg to about 0.7 mg for a total nominal fill weight in the pocket of 7 mg; or (ii) from about 0.024 mg to about 0.42 mg for a total nominal fill weight in the pocket of 7 mg; or (iii) about 0.441 mg for a total nominal fill weight in the pocket of about 7 mg.
Clause 7: The powder according to any one of clauses 1-5, wherein a nominal dose of carboxamide compound of formula II

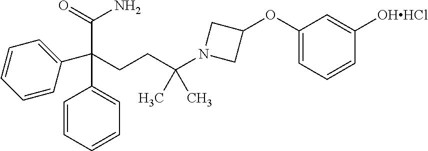

per nominal pocket fill comprises: (i) from about 0.019 mg to about 0.757 mg for a total nominal fill weight in the pocket of about 7 mg; or (ii) about 0.026 mg to about 0.454 mg nominal fill weight in the pocket of about 7 mg; or (iii) about 0.477 mg nominal fill weight in the pocket of about 7 mg.
Clause 8: The powder according to any one of clauses 1-7, wherein the excipient comprises: (i) monosaccharides, disaccharides, oligosaccharides or polysaccharides, or a combination thereof; (ii) glucose, arabinose, lactose, sucrose, maltose, dextrans, or lactose, or a combination thereof; (iii) lactose monohydrate; (iv) phosphatidylcholine, 1-leucine, mannitol, or magnesium stearate; or (v) menthol, levomenthol, saccharin, or saccharin sodium, or a combination thereof.
Clause 9: The powder according to clause 8, wherein the lactose monohydrate is in the powder in: (i) an amount from about 5.86 mg to about 6.98 mg; or (ii) an amount from about 5.86 mg to about 6.98 mg.
Clause 10: The powder according to clause 8 or clause 9, wherein the lactose monohydrate comprises particles having a mass-median-diameter D50 from about 60 μm to about 80 μm.
Clause 11: The powder according to any one of clauses 1-10, wherein the excipient comprises from about 10% by weight to about 99.5% by weight of the formulation.
Clause 12: The powder according to any one of clauses 1-11, wherein: (i) the dry powder comprises fine particles and coarse particles and the ratio between the fraction of fine particles and the fraction of coarse particles is between about 0.25 and about 100; or (ii) the carboxamide compound of formula I or formula II having a D90 particle size from about 5 μm to about 10 μm.
Clause 13: The powder according to any one of clauses 1-12, wherein about 7% by weight of the carboxamide compound of formula I comprises a fine particle mass of about 128 μg for a nominal dose of carboxamide compound of about 441 μg.
Clause 14: The powder according to any one of clauses 1-13, wherein: (i) the carboxamide compound of formula I has a mean fine particle fraction of from about 31% to about 37% total impactor recovery; or (ii) the carboxamide compound of formula I has a particle mass from about 110 μg to about 160 μg.
Clause 15: The powder according to any one of clauses 1-14, wherein the powder is a fine powder having a fine particle dose of from about 20 μg to about 160 μg; or from about 161 μg to about 245 μg.
Clause 16: A powder formulation for use in a dry powder inhaler, the powder comprising a carboxamide compound of formula I

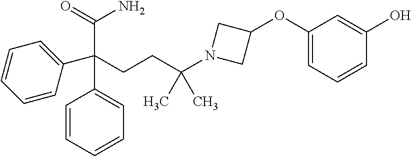 [SMILES: CC(C)(CCC(C(N)=O)(c1ccccc1)c1ccccc1)N1CC(Oc2cccc(O)c2)C1]

or a pharmaceutically acceptable salt thereof, a pharmaceutically acceptable excipient, and a long acting β2 adrenoceptor agonist and optionally a corticosteroid.
Clause 17: The powder formulation according to clause 16, wherein the carboxamide compound is 5-[3-(3-Hydroxyphenoxy) azetidin-1-yl]-5-methyl-2, 2-diphenylhexanamide hydrochloride of formula II

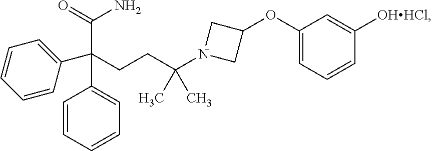

the β2 adrenoceptor agonist comprises salmeterol xinafoate and the corticosteroid comprises fluticasone propionate.
Clause 18: The powder formulation according to clause 16 or clause 17, wherein the powder contains from about 0.8% by weight to about 10% by weight of the carboxamide compound of formula I.
Clause 19: The powder formulation according to any one of clauses 16-18, wherein the excipient comprises: (i) monosaccharides, disaccharides, oligosaccharides or polysaccharides, or a combination thereof; (ii) glucose, arabinose, lactose, sucrose, maltose, dextrans, or lactose, or a combination thereof; (iii) lactose monohydrate; (iv) phosphatidylcholine, 1-leucine, mannitol, or magnesium stearate; or (v) menthol, levomenthol, saccharin, or saccharin sodium, or a combination thereof.
Clause 20: The powder formulation according to any one of clauses 16-19, wherein the dry powder inhaler comprises a plurality of pockets, each pocket configured to comprise at least two blend layers, the at least two blend layers comprising: (i) a first blend containing from about 0.8% to about 10% by weight of the carboxamide compound of formula I filled to a nominal weight of about 7 mg in the pocket and a second blend containing about 2% by weight of fluticasone propionate and about 0.4% by weight salmeterol filled to a nominal 12.5 mg in the pocket, both blends filled to a total nominal pocket fill weight of from about 18.8 mg to about 19.5 mg; or (ii) a first blend containing from about 0.056 mg to about 0.441 mg of the carboxamide compound of formula I and from about 5.86 mg to about 6.94 mg of lactose monohydrate filled to a nominal fill weight of from about 6.3 mg to about 7.0 mg, and a second blend containing from about 0.05 mg salmeterol, about 0.250 mg of fluticasone propionate and about 12.2 mg lactose monohydrate, both blends filled to a total pocket fill weight of from about 18.8 mg to about 19.5 mg for a total pocket fill weight of from about 18.8 mg to about 19.5 mg; or (iii) a first blend containing from about 0.87 mg to about 10.82 mg of the carboxamide compound of formula II filled to a nominal 7 mg of powder added to the pocket, and a second blend containing from about 2.00 mg of fluticasone propionate, from about 0.58 mg of salmeterol xinafoate, both blends filled to a total nominal fill weight of from about 18.8 mg to about 19.5 mg, or (iv) a first blend from about 0.061 mg to about 0.477 mg of the carboxamide compound of formula II and from about 5.823 mg to about 6.939 mg of lactose mononhydrate for a nominal fill weight of about 7.0 mg, and a second blend from about 0.250 mg of fluticasone propionate and about 0.073 mg of salmeterol xinafoate and about 12.177 mg lactose monohydrate for a nominal fill weight of about 12.5 mg, both blends filled to a total nominal fill weight of from about 18.8 to about 19.5.
Clause 21: The powder formulation according to any one of claims 16-20, wherein the dry powder inhaler comprises a plurality of pockets, each pocket configured to comprise at least one blend layer, the blend layer comprising: (i) a mixture of a first blend containing from about 0.8% to about 10% by weight of the carboxamide compound of formula I filled to a nominal weight of about 7 mg in the pocket and a second blend containing about 2% by weight of fluticasone propionate and about 0.4% by weight salmeterol filled to a nominal fill weight of about 12.5 mg in the pocket; or (ii) a mixture of a first blend of from about 0.29% w/w to about 3.59% w/w of the carboxamide compound of formula I and a second blend containing about 0.26% w/w of salmeterol and from about 1.28% w/w to about 1.33% w/w fluticasone propionate for a total nominal fill weight in the pocket from about 18.8 mg to about 19.5 mg; or (iii) a mixture of from about 0.056 mg to about 0.441 mg of carboxamide compound of formula I, from about 0.250 mg of fluticasone propionate, about 0.05 mg salmeterol and from about 18.06 mg to about 19.144 mg of lactose monohydrate for a total fill weight in the pocket of from about 18.8 mg to about 19.5 mg; or (iv) a mixture of a first blend containing from about 0.31% w/w to about 3.88% w/w of carboxamide compound of formula II, and a second blend containing from about 1.28% w/w to about 1.33% w/w of fluticasone propionate, from about 0.37% w/w to about 0.39% w/w of salmeterol xinafoate for a total nominal fill weight in the pocket of from about 18.8 mg to about 19.5 mg; or (v) a mixture of from about 0.061 mg to about 0.757 mg of carboxamide compound of formula II, about 0.250 mg fluticasone propionate and about 0.73 mg of salmeterol xinafoate and form about 18.42 mg to about 19.12 mg of lactose monohydrate for a total fill weight in the pocket of from about 18.8 mg to about 19.5 mg.
Clause 22: The powder formulation according to any one of clauses 19-21, wherein the lactose monohydrate comprises particles having a mass-median-diameter D50 from about 60 μm to about 80 μm.
Clause 23: The powder formulation according to any one of clauses 16-22, wherein the carboxamide compound of formula I or formula II has a particle size from about 5 μm to about 10 μm, the salmeterol or salmeterol xinafoate has a particle size from about 5 μm to about 10 μm, and the fluticasone propionate has a particle size from about 5 μm to about 10 μm.
Clause 24: The powder formulation according to any one of clauses 16-23, wherein the formulation is a fine powder having a fine powder dose that varies from about 20 μg to about 160 μg; or from about 161 μg to about 245 μg.
Clause 25: A method of making a powder formulation for use in a dry powder inhaler, the method comprising mixing a powder formulation comprising from about 0.01% by weight to about 90% by weight of a carboxamide compound of formula I

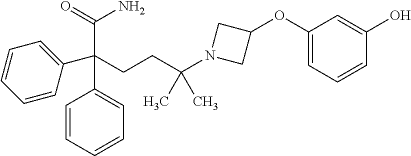 [SMILES: CC(C)(CCC(C(N)=O)(c1ccccc1)c1ccccc1)N1CC(Oc2cccc(O)c2)C1]

or a pharmaceutically acceptable salt thereof with an excipient.
Clause 26: The method of making the powder formulation according to clause 25, wherein the carboxamide compound is a compound of formula II

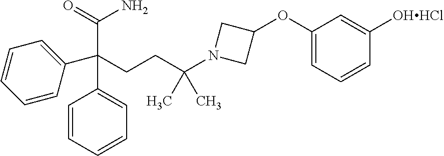

Clause 27: The method of making the powder formulation according to any one of clause or clause 26, wherein the carboxamide compound is 5-[3-(3-Hydroxyphenoxy)azetidin-1-yl]-5-methyl-2,2-diphenylhexanamide hydrochloride.
Clause 28: The method of making the powder formulation according to any one of clauses 25-27, wherein the excipient comprises: (i) monosaccharides, disaccharides, oligosaccharides or polysaccharides, or a combination thereof; (ii) glucose, arabinose, lactose, sucrose, maltose, dextrans, or lactose, or a combination thereof; (iii) lactose monohydrate; (iv) phosphatidylcholine, 1-leucine, mannitol, or magnesium stearate; or (v) menthol, levomenthol, saccharin, or saccharin sodium, or a combination thereof.
Clause 29: The method of making the powder formulation according to any one of clauses 25-28, the method comprising mixing a powder comprising a carboxamide compound of formula I

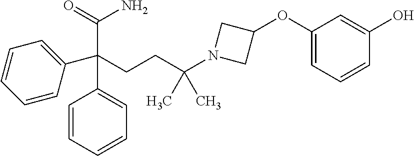 [SMILES: CC(C)(CCC(C(N)=O)(c1ccccc1)c1ccccc1)N1CC(Oc2cccc(O)c2)C1]

or a pharmaceutically acceptable salt thereof, with a pharmaceutically acceptable excipient to form a mixture and adding a long acting β2 adrenoceptor agonist or a corticosteroid or both to the mixture.
Clause 30: The method of making the powder formulation according to any one of clauses 25-29, wherein the carboxamide compound is 5-[3-(3-Hydroxyphenoxy) azetidin-1-yl]-5-methyl-2, 2-diphenylhexanamide hydrochloride of formula II

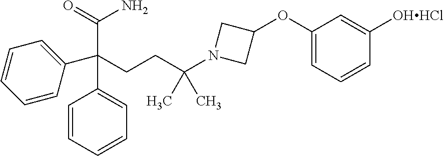

the β2 adrenoceptor agonist comprises salmeterol xinafoate and the corticosteroid comprises fluticasone propionate.

Additional features and advantages of various embodiments will be set forth in part in the description that follows, and in part will be apparent from the description, or may be learned by practice of various embodiments. The objectives and other advantages of various embodiments will be realized and attained by means of the elements and combinations particularly pointed out in the description and appended claims.

### BRIEF DESCRIPTION OF THE DRAWING

In part, other aspects, features, benefits and advantages of the embodiments will be apparent with regard to the following description, appended claims and accompanying drawings where:

FIG. 1 is an individual value plot of assay values taken at an initial time, 1 month and 3 months at 25° C. and 60% relative humidity (RH), 30° C. and 65% RH, 40° C. and 75% RH;

FIG. 2 illustrates the emitted dose of batches of MGR002 and MGR003 products as described herein;

FIG. 3 illustrates the aerosolization performance of MGR002 and MGR003 products measured by fine particle mass (FPM);

FIG. 4 illustrates equivalence criteria for carboxamide hydrochloride of formula II in batches of MGR002 and MGR003 expressed in FPM;

FIG. 5 illustrates the stability of MGR002 and MGR003 products expressed in FPM percent total impactor recovery (TIR) from a next generation inhaler (NGI);

FIG. 6 illustrates the stability of MGR002 and MGR003 products expressed in FPM;

FIG. 7 illustrates results from a three-month stability study performed on a 23.8 μg nominal dose of the MGR002 product. The initial time point for the 23.8 μg product produced an FPM value of 5.4 μg. Results showed a stability drop to 4.7 μg after three months at 25° C./60% RH. After three months at an accelerated storage condition of 40° C./75% RH, the FPM dropped to 3.8 μg; and

FIG. 8 illustrates results from a three-month stability study performed on a 32.9 μg nominal dose of the MGR002 product. The initial time point for the 32.9 μg product produced an FPM value of 7.7 μg. Results showed a stability drop to 7.1 μg after three months at 25° C./60% RH and 5.7 μg at 40° C./75% RH.

It is to be understood that the figures are not drawn to scale. Further, the relation between objects in a figure may not be to scale, and may in fact have a reverse relationship as to size. The figures are intended to bring understanding and clarity to the structure of each object shown, and thus, some features may be exaggerated in order to illustrate a specific feature of a structure.

### DETAILED DESCRIPTION

The present disclosure may be understood more readily by reference to the following detailed description of the disclosure presented in connection with the accompanying drawings, which together form a part of this disclosure. It is to be understood that this disclosure is not limited to the specific formulations, methods, conditions or parameters described and/or shown herein, and that the terminology used herein is for the purpose of describing particular embodiments by way of example only and is not intended to be limiting of the claimed disclosure. The following description is presented to enable any person skilled in the art to make and use the present disclosure.

For the purposes of this specification and appended claims, unless otherwise indicated, all numbers expressing quantities of ingredients, percentages or proportions of materials, reaction conditions, and other numerical values used in the specification and claims, are to be understood as being modified in all instances by the term “about.” Accordingly, unless indicated to the contrary, the numerical parameters set forth in the following specification and attached claims are approximations that may vary depending upon the desired properties sought to be obtained by the present application. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the present application are approximations, the numerical values set forth in the specific examples are reported as precisely as possible. Any numerical value, however, inherently contains certain errors necessarily resulting from the standard deviation found in their respective testing measurements. Moreover, all ranges disclosed herein are to be understood to encompass any and all sub ranges subsumed therein. For example, a range of “1 to 10” includes any and all sub ranges between (and including) the minimum value of 1 and the maximum value of 10, that is, any and all sub ranges having a minimum value of equal to or greater than 1 and a maximum value of equal to or less than 10, e.g., 5.5 to 10.

In this application, all temperatures are set forth in Celsius degrees; and, unless otherwise indicated, all parts and percentages are by weight.

In this application, the section headings below should not be restricted and can be interchanged with other section headings.

### Definitions

It is noted that, as used in this specification and the appended claims, the singular forms “a”, “an”, and “the” include plural referents unless expressly and unequivocally limited to one referent. Thus, for example, reference to “an excipient” includes one, two, three or more excipients.

Ranges may be expressed in this application as from “about” or “approximately” one particular value and/or to “about” or “approximately” another particular value. When such a range is expressed, another embodiment includes from the one particular value and/or to the other particular value.

The terms “having”, “containing”, “including”, “comprising” and the like are used herein to refer to open ended terms that indicate the presence of stated elements or features, but do not preclude additional elements or features.

MGR001 refers to a dry powder for inhalation in, for example, a CRC749 dry powder inhaler device, wherein the actives are fluticasone propionate and salmeterol xinafoate in a ratio of 250 μg/50 μg. In some embodiments, the fluticasone propionate and salmeterol xinafoate are in a ratio of 500 μg/50 μg or 100 μg/50 μg. CRC749 dry powder inhaler has been described in U.S. Pat. Nos. 9,399,103 and/or 9,561,336, incorporated herein by reference as if set forth in full.

MGR002 refers to a dry powder formulation for inhalation (441 μg) in, for example, a CRC749 dry powder inhaler device, wherein the active ingredient is 5-[3-(3-Hydroxyphenoxy) azetidin-1-yl]-5-methyl-2, 2-diphenylhexanamide hydrochloride, a carboxamide hydrochloride compound of formula II.

MGR003 refers to dry powder formulation for inhalation in, for example, a CRC749 dry powder inhaler device, wherein the actives are a fluticasone propionate, salmeterol xinafoate and carboxamide hydrochloride of formula II in a ratio of 250 μg/50 μg/441 μg.

FORM004 refers to dry powder formulation for inhalation in, for example, a CRC749 dry powder inhaler device, wherein the actives are salmeterol or salmeterol xinafoate and carboxamide compound of formula I or carboxamide hydrochloride of formula II in a ratio of 50 μg/441 μg.

FORM005 refers to dry powder formulation for inhalation in, for example, a CRC749 dry powder inhaler device, wherein the actives are fluticasone propionate and carboxamide compound of formula I or carboxamide hydrochloride of formula II in a ratio of 250 μg/441 μg.

The term “excipient” is used herein to describe an ingredient other than the hydrochloride salt of this application. The choice of excipient, will to a large extent, depend on the particular mode of administration, the effect of the excipient on solubility and stability, and the nature of the dosage form.

The term, “label claim” (LC) refers to the drug content reported on the product label as being present in the dosage form.

The term “active pharmaceutical ingredient” (API) as used herein, includes any substance (i.e., compound or composition of matter) which, when administered to an organism (human or animal) induces a desired pharmacologic and/or physiologic effect by local and/or systemic action. The term therefore encompasses substances traditionally regarded as actives, drugs or bioactive agents, as well as biopharmaceuticals (for example, peptides, hormones, nucleic acids, gene constructs) typically employed to treat a number of conditions which is defined broadly to encompass diseases, disorders, infections, or the like. Exemplary APIs include, without limitation, antibiotics, antivirals, H₂-receptor antagonists, 5HT₁ agonists, 5HT₃ antagonists, COX2-inhibitors, steroids (e.g., prednisone, prednisolone, dexamethasone) APIs used in treating psychiatric conditions such as depression, anxiety, bipolar condition, tranquilizers, APIs used in treating metabolic conditions, anticancer APIs, APIs used in treating neurological conditions such as epilepsy and Parkinson's Disease, APIs used in treating cardiovascular conditions, non-steroidal anti-inflammatory APIs, APIs used in treating Central Nervous System conditions, or APIs employed in treating hepatitis. In this application, the API can be muscarinic M₃ receptor agonists or anticholinergic agents, β2-adrenoceptor agonists, compounds having a dual muscarinic antagonist and β2-agonist activity and glucocorticoid receptor agonists or corticosteroids.

In some embodiments, the API is ipratropium, tiotropium, oxitropium, trospium, aclidiniums, perenzepine, telenzepine, ephedrine, adrenaline, isoprenaline, metaproterenol, phenylephrine, phenylpropanolamine, pirbuterol, reproterol, rimiterol, isoetharine, carmoterol, albuterol, terbutaline, bambuterol, fenoterol, salbutamol, tulobuterol formoterol, salmeterol, prednisone, prednisolone, flunisolide, triamcinolone acetonide, beclomethasone, budesonide, fluticasone, ciclesonide, mometasone as well as salts and/or solvates thereof.

The term “hydrochloride salt” includes the hydrochloride salt of 5-[3-(3-hydroxyphenoxy)azetidin-1-yl]-5-methyl-2,2-diphenylhexanamide and its derived forms. The hydrochloride salt is a valuable pharmaceutically active compound, which is suitable for the therapy and prophylaxis of numerous disorders in which muscarinic receptors are involved or in which antagonism of this receptor may induce benefit, in particular the allergic and non-allergic airways diseases (e.g., asthma, COPD) but also in the treatment of other diseases such as Inflammatory Bowel Disease, Irritable Bowel Disease, diverticular disease, motion sickness, gastric ulcers, radiological examination of the bowel, symptomatic treatment of BPH (benign prostatic hyperplasia), NSAID induced gastric ulceration, urinary incontinence (including urgency, frequency, urge incontinence, overactive bladder, nocturia and lower urinary tract symptoms), cycloplegia, mydriatics and Parkinson's disease. The hydrochloride salt of this application can be administered according to this application to animals, in many instances, to mammals, and in particular to humans, as pharmaceutical for therapy and/or prophylaxis.

A “therapeutically effective amount” or “effective amount” is such that when administered, the carboxamide of this application, results in alteration of the biological activity, such as, for example, in the treatment of diseases, disorders and conditions in which the M₃ receptor is involved. The dosage administered to a patient can be as single or multiple doses depending upon a variety of factors, including the drug's administered pharmacokinetic properties, the route of administration, patient conditions and characteristics (sex, age, body weight, health, size, etc.), and extent of symptoms, concurrent treatments, frequency of treatment and the effect desired. In some embodiments, the formulation is designed for immediate release. In other embodiments, the formulation is designed for sustained release. In other embodiments, the formulation comprises one or more immediate release surfaces and one or more sustained release surfaces.

A “pharmaceutically acceptable carrier” is meant as a material that is not biologically or otherwise undesirable, e.g., the material may be administered to a subject without causing any undesirable biological effects or interacting in a deleterious manner with any of the other components of the pharmaceutical composition in which it is contained.

The term “pharmaceutically acceptable salt” comprises inorganic and organic salts. Examples of organic salts may include formate, acetate, trifluoroacetate, propionate, butyrate, lactate, citrate, tartrate, malate, maleate, succinate, methanesulfonate, benzenesulfonate, xinafoate, pamoate, and benzoate. Examples of inorganic salts may include fluoride chloride, bromide, iodide, phosphate, nitrate and sulphate.

The term “coarse” refers to a substance having a size of one or few hundred microns. Coarse particles is expressed in terms of mass diameter. The particles have a normal (Gaussian) distribution which is defined in terms of the volume or mass median diameter (VMD or MMD) which corresponds to the volume or mass diameter (D₅₀) of 50 percent by weight of the particles, and, optionally, in terms of volume or mass diameter of 10% and 90% of the particles, respectively.

Another common approach to define the particle size distribution is to cite three values: (i) the median diameter d(0.5) which is the diameter where 50% of the distribution is above and 50% is below; (ii) d(0.9), where 90% of the distribution is below this value; (iii) d(0.1), where 10% of the distribution is below this value.

The expression “good homogeneity” refers to a powder wherein, upon mixing, the uniformity of distribution of a component, expressed as coefficient of variation (CV) also known as relative standard deviation (RSD), is less than 5.0%. It is usually determined according to known methods, for instance by taking samples from different parts of the powder and testing the component by HPLC or other equivalent analytical methods.

The expression “respirable fraction” refers to an index of the percentage of active particles which would reach the lungs in a patient. The respirable fraction is evaluated using a suitable in vitro apparatus such as Andersen Cascade Impactor (ACI), Multi Stage Liquid Impinger (MSLI) or Next Generation Impactor (NGI), according to procedures reported in common Pharmacopoeias, in particular in the European Pharmacopeia (Eur. Ph.) 7.3, 7th Edition, which is incorporated herein by reference in its entirety.

The term “dry powder inhaler” (DPI), refers to a device that delivers medication to the lungs in the form of a dry powder. DPIs are commonly used to treat respiratory diseases such as asthma, bronchitis, emphysema and COPD. DPIs can be divided into two basic types: (i) single dose inhalers, for the administration of single subdivided doses of the active compound; each single dose is usually filled in a capsule; and (ii) multidose inhalers pre-loaded with quantities of active principles sufficient for longer treatment cycles.

The term “solvate” is used herein to describe a molecular complex comprising the hydrochloride salt of this disclosure and a stoichiometric amount of one or more pharmaceutically acceptable solvent molecules, for example, ethanol. Examples of solvents that form solvates include, but are not limited to, water, isopropanol, ethanol, methanol, DMSO, ethyl acetate, acetic acid, and ethanolamine.

The term “hydrate” refers to the aggregate or complex where the solvent molecule is water. The solvent may be inorganic solvents such as, for example, water in which case the solvate may be a hydrate. Alternatively, the solvent may be an organic solvent, such as ethanol. The compound of the disclosure may be a true solvate, while in other cases, the compound of the disclosure may merely retain adventitious water or be a mixture of water plus some adventitious solvent.

The term “treatment” includes references to curative, palliative and prophylactic treatment.

Reference will now be made in detail to certain embodiments of this application, examples of which are illustrated in the accompanying drawings. While the drawings will be described in conjunction with the illustrated embodiments, it will be understood that they are not intended to limit this application to those embodiments.

### Powder Formulations

A powder formulation for use in a dry powder inhaler is provided. In addition to an excipient, the powder formulation can include a single pharmaceutically active ingredient, or a combination of two or three active ingredients. In some embodiments, as the single pharmaceutically active ingredient, the powder formulation comprises, consists essentially of or consists of a muscarinic M₃ receptor agonist or an anticholinergic agent, a β2-adrenoceptor agonist, or a compound having a dual muscarinic antagonist and β2-agonist activity or a glucocorticoid receptor agonist. More specifically, the single pharmaceutical active ingredient comprises, consists essentially of or consists of ipratropium, tiotropium, oxitropium, trospium, aclidiniums, perenzepine, telenzepine, ephedrine, adrenaline, isoprenaline, metaproterenol, phenylephrine, phenylpropanolamine, pirbuterol, reproterol, rimiterol, isoetharine, carmoterol, albuterol, terbutaline, bambuterol, fenoterol, salbutamol, tulobuterol formoterol, salmeterol, prednisone, prednisolone, flunisolide, triamcinolone acetonide, beclomethasone, budesonide, fluticasone, ciclesonide, mometasone as well as salts and/or solvates thereof.

In certain aspects, the pharmaceutically active ingredient can be selected from numerous classes of medications such as, for example, glucocorticoid receptor agonists, PDE inhibitors in particular PDE4 inhibitors, sodium cromoglycate, muscarinic M₃ receptor antagonists or anticholinergic agents, β2-adrenoceptor agonists, compounds having a dual muscarinic antagonist and β2-agonist activity, anti-tumour necrosis factor (anti-TNF-α) agents, adenosine A2a receptor agonists and A2b antagonists, histamine H₃ antagonists and H₄ antagonists, modulators of prostaglandin D2 including DP1 antagonists, DP2 antagonists and inhibitors of haematopoietic prostaglandin D synthase (hPGDS), modulators of the NFκβ pathway such as IKK inhibitors, modulators of cytokine signalling pathways such as p38 MAP kinases, PI3 kinases, JAK kinases, syk kinase, EGFR, MK-2, fyn kinases or ITK.

According to another embodiment of the present application, the pharmaceutically active ingredient may be selected from: (i) muscarinic M₃ receptor agonists or anticholinergic agents such as ipratropium, tiotropium, oxitropium, trospium, aclidinium, perenzepine, telenzepine and other muscarinic agonists such as those described in WO 03/035599, WO 2007/034325, WO 08/035157, or WO 2009/034432, as well as salts and/or solvates thereof; (ii) β2-adrenoceptor agonists such as ephedrine, adrenaline, isoprenaline, metaproterenol, phenylephrine, phenylpropanolamine, pirbuterol, reproterol, rimiterol, isoetharine, carmoterol, albuterol, terbutaline, bambuterol, fenoterol, salbutamol, tulobuterol formoterol, salmeterol, and other β2-agonists such as e.g., those described in WO 04/032921, WO 05/080313, WO 05/080324, WO 05/090287, WO 05/092840, and WO 2007/010356 as well as salts and/or solvates thereof; (iii) compounds having a dual muscarinic antagonist and β2-agonist activity such as e.g., those described in WO 2007/107828 or WO 2008/041095; (iv) glucocorticoid receptor agonists such as prednisone, prednisolone, flunisolide, triamcinolone acetonide, beclomethasone, budesonide, fluticasone, ciclesonide, mometasone as well as salts and/or solvates thereof; and dual or triple combinations thereof.

In some aspects, the powder formulation comprising from about 0.01% by weight to about 90% by weight of a carboxamide compound of formula I

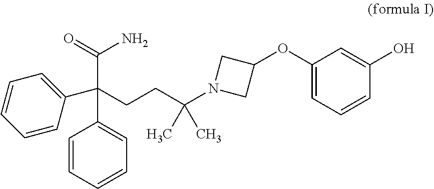 [SMILES: CC(C)(CCC(C(N)=O)(c1ccccc1)c1ccccc1)N1CC(Oc2cccc(O)c2)C1]

or C₂₈H₃₂N₂O₃ or a pharmaceutically acceptable salt thereof and an excipient.

In other aspects, the pharmaceutically acceptable salt of the carboxamide compound of formula I is a compound of formula II

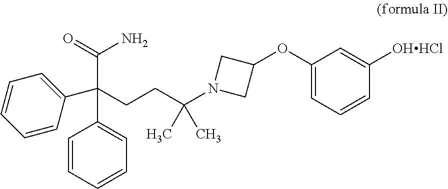

also known as 5-[3-(3-Hydroxyphenoxy)azetidin-1-yl]-5-methyl-2,2-diphenylhexanamide hydrochloride (carboxamide hydrochloride) or C₂₈H₃₂N₂O₃·HCl.

It has been found that the hydrochloride salt of this application is an antagonist of the M₃ receptor, which is particularly useful for the treatment of M₃-mediated diseases and/or conditions, and shows good potency, in particular when administered via the inhalation route. The hydrochloride salt of this application is particularly suitable for an administration by the inhalation route. In particular, the hydrochloride salt of this application can be formulated for an administration using a dry powder inhaler.

The hydrochloride salt of this application can also be administered intranasally or by inhalation, typically in the form of a dry powder (either alone, as a mixture, for example, in a dry blend with lactose, or as a mixed component particle, for example, mixed with phospholipids, such as phosphatidylcholine) from a dry powder inhaler or as an aerosol spray from a pressurized container, pump, spray, atomizer (in some aspects, an atomizer using electro hydrodynamics to produce a fine mist), or nebulizer, with or without the use of a suitable propellant, such as 1,1,1,2-tetrafluoroethane or 1,1,1,2,3,3,3-heptafluoropropane. For intranasal use, the powder may comprise a bioadhesive agent, for example, chitosan or cyclodextrin. When used with a propellant, the carboxide hydrochloride compound of formula II further comprises propellants, including, but not limited to, hydrofluoroalkane (HFA), such as chlorodifluoromethane, trifluoromonofluoroethane, chlorodifluoroethane, difluoroethane, heptafluoropropane, or a combination thereof.

The pressurized container, pump, spray, atomizer, or nebulizer contains a solution or suspension of the compound(s) of this application comprising, for example, ethanol, aqueous ethanol, or a suitable alternative agent for dispersing, solubilizing, or extending release of the active, a propellant(s) as solvent and an optional surfactant, such as sorbitan trioleate, oleic acid, or an oligolactic acid.

Prior to use in a dry powder or suspension formulation, the drug product is micronized to a size suitable for delivery by inhalation (typically less than 5 microns). This may be achieved by any appropriate comminuting method, such as spiral jet milling, fluid bed jet milling, supercritical fluid processing to form nanoparticles, high pressure homogenization, or spray drying.

In some embodiments, the 5-[3-(3-Hydroxyphenoxy)azetidin-1-yl]-5-methyl-2,2-diphenylhexanamide or carboxamide compound of formula I or 5-[3-(3-Hydroxyphenoxy)azetidin-1-yl]-5-methyl-2,2-diphenylhexanamide hydrochloride or carboxamide hydrochloride of formula II are in a dose of about 40 to about 800 μg, the salmeterol xinafoate is in a dose of about 72.5 μg, and the fluticasone propionate is in a dose of about 100 to about 500 μg.

The 5-[3-(3-Hydroxyphenoxy)azetidin-1-yl]-5-methyl-2,2-diphenylhexanamide or carboxamide compound of formula I or 5-[3-(3-Hydroxyphenoxy)azetidin-1-yl]-5-methyl-2,2-diphenylhexanamide hydrochloride or carboxamide hydrochloride of formula II are in a dose of from about 40 to about 800 μg. In some embodiments, the 5-[3-(3-Hydroxyphenoxy)azetidin-1-yl]-5-methyl-2,2-diphenylhexanamide hydrochloride is in a dose of from about 40 to about 700 μg, from about 40 to about 600 μg, from about 40 to about 500 μg, from about 40 to about 400 μg, from about 40 to about 300 μg, from about 40 to about 200 μg, from about 40 to about 100 μg, from about 100 to about 800 μg, from about 100 to about 700 μg, from about 100 to about 600, μg from about 100 to about 500 μg, from about 100 to about 400 μg, from about 100 to about 300 μg, from about 100 to about 200 μg, or from about 100 to about 150 μg.

In some embodiments, the 5-[3-(3-Hydroxyphenoxy)azetidin-1-yl]-5-methyl-2,2-diphenylhexanamide or carboxamide compound of formula I or 5-[3-(3-Hydroxyphenoxy) azetidin-1-yl]-5-methyl-2,2-diphenylhexanamide hydrochloride or carboxamide hydrochloride of formula II are in a dose of from about 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690 to about 700 μg.

In some embodiments, the 5-[3-(3-Hydroxyphenoxy)azetidin-1-yl]-5-methyl-2,2-diphenylhexanamide or carboxamide compound of formula I or 5-[3-(3-Hydroxyphenoxy)azetidin-1-yl]-5-methyl-2,2-diphenylhexanamide hydrochloride or carboxamide hydrochloride of formula II are in an amount from about 0.01 to about 99 wt. % of the formulation and/or the at least first layer. In some embodiments, the -[3-(3-Hydroxyphenoxy)azetidin-1-yl]-5-methyl-2,2-diphenylhexanamide hydrochloride is in an amount from about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84 85, 86, 87, 88, 89, 90, 91, 92, 93, 94, 95, 96, 97, 98 or 99 wt. % of the formulation and/or the at least first layer.

The 5-[3-(3-Hydroxyphenoxy) azetidin-1-yl]-5-methyl-2,2-diphenylhexanamide or the carboxamide compound of formula I or 5-[3-(3-Hydroxyphenoxy)azetidin-1-yl]-5-methyl-2,2-diphenylhexanamide hydrochloride or the carboxamide hydrochloride of formula II have a particle size of from about less than 10 μm to about less than 5 μm. In some embodiments, the 5-[3-(3-Hydroxyphenoxy)azetidin-1-yl]-5-methyl-2,2-diphenylhexanamide or the carboxamide compound of formula I or 5-[3-(3-Hydroxyphenoxy)azetidin-1-yl]-5-methyl-2,2-diphenylhexanamide hydrochloride or the carboxamide hydrochloride of formula II have a particle size of from about less than 1 μm, 2 μm, 3 μm, 4 μm, 5 μm, less than 6 μm, less than 7 μm, less than 8 μm, less than 9 μm, to about less than 10 μm.

Excipients that are useful in the monotherapy powder formulation of this application can be any physiologically acceptable excipient which can be used in the context of the inhalable formulation of this application. For example, the excipient may be selected from monosaccharides, disaccharides, oligo- and polysaccharides. In some embodiments, the excipient can include, but is not limited to, monosaccharides such as galactose, mannose, sorbose; disaccharides such as lactose, sucrose and trehalose and the like; polysaccharides such as starch, raffinose, dextran and the like; sugar alcohols (including glycerol, erythritol, arabitol, xylitol, sorbitol, mannitol); glycols (including ethylene glycol, propylene glycol, polyethylene glycol, polypropylene glycol); cellulose-like polymers (including hydroxy cellulose, hydroxy propyl cellulose); insoluble additives (crystalline cellulose, chitosan, calcium carbonate, talc, titanium oxide) or silica (silicon oxide), and mixtures thereof. In some aspects, the excipient is lactose, in particular, lactose monohydrate.

In other embodiments, the powder formulation according to this application may further be made of a mixture of components comprising a component as described above together with other components selected from, for example, phospholipids such as phosphatidylcholine, performance modifier such as 1-leucine, mannitol, or magnesium stearate. Suitable flavors, such as menthol and levomenthol, or sweeteners, such as saccharin or saccharin sodium, may be added to the powder formulations intended for inhaled/intranasal administration.

In some embodiments, the excipient comprises from about 0.01 to about 99.9 wt. % of the monotherapy formulation. In some embodiments, the excipient comprises from about 0.1 to about 99 wt. %, from about 1 to about 99 wt. %, from about 10 to about 99 wt. %, from about 20 to about 99 wt. %, from about 30 to about 99 wt. %, from about 40 to about 99 wt. %, from about 50 to about 99 wt. %, from about 60 to about 99 wt. %, from about 70 to about 99 wt. %, from about 80 to about 99 wt. %, from about 90 to about 99 wt. %, from about 95 to about 99 wt. %, or from about 97 to about 99 wt. % of the monotherapy formulation.

In some embodiments, the excipient comprises from about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 99.9 wt. % of the monotherapy formulation. In other embodiments, the excipient comprises from about 10% by weight to 99.5% by weight of the formulation.

In various embodiments, the excipient is a powder and has an average particle size of from about 1, 2, 3, 4, 5, 6, 7, 8, 9, 10 to about 200 μm. In some embodiments, the excipient has an average particle size of from about 1, 2, 3, 4, 5, 6, 7, 8, 9, 10 to about 150 μm, from about 10 to about 100 μm, from about 10 to about 75 μm, from about 10 to about 50 μm, from about 25 to about from about 150 μm, from about 25 to about 100 μm, from about 25 to about 75 μm, or from about 25 to about 50 μm. In some embodiments, the excipient has an average particle size of from about 1, 10, 20, 30, 40, 50, 60, 70, 80, 90, 100, 110, 120, 130, 140, 150, 160, 170, 180, 190 to about 200 μm. The excipient can be a coarse powder or a fine powder or a combination of both.

Useful excipients include, in some embodiments: (i) monosaccharides, disaccharides, oligosaccharides or polysaccharides or a combination thereof; (ii) glucose, arabinose, lactose, sucrose, maltose, dextrans, lactose or a combination thereof; (iii) lactose monohydrate; or (iv) phosphatidylcholine, 1-leucine, mannitol, or magnesium stearate; flavors such as menthol, levomenthol, saccharin, saccharin sodium or a combination thereof.

In various aspects, the excipient powder that may be used with the powder formulation is made of particles having an average size that is less than 200 μm, in some aspects, less than 100 μm. In certain applications, the excipient powder is made of particles having an average particle size comprising from about 10 μm, 20 μm, 30 μm, 40 μm, 50 μm, 60 μm, 70 μm to about 80 μm, still, in other aspects, from about 15 μm, 20 μm, 25 μm, 30 μm, 35 μm, 40 μm, 45 μm, 50 μm, 55 μm, to about 60 μm. In certain aspects, the powder formulation contains lactose monohydrate in an amount from about 10% weight by weight (w/w), 20% w/w, 30% w/w, 40% w/w, 50% w/w, 60% w/w, 70% w/w, 80% w/w, 90% w/w to about 90.5% w/w. In yet other aspects, the lactose monohydrate excipient comprises particles having a mass-median-diameter D50 from about 60 μm, 70 μm to about 80 μm.

In certain embodiments, the dry powder formulation of this application comprises, consists essentially of, or consists of fine particles and coarse particles and the ratio between the fraction of fine particles and the fraction of coarse particles is between 1 and 100. In other aspects, the carboxamide compound of formula I or a pharmaceutically acceptable salt thereof has a particle size from about 5 μm to about 10 μm. In some embodiments, the carboxamide compound of formula I or the 5-[3-(3-Hydroxyphenoxy)azetidin-1-yl]-5-methyl-2,2-diphenylhexanamide hydrochloride have a particle size of from about less than 5 μm, less than 6 μm, less than 7 μm, less than 8 μm, less than 9 μm, to about less than 10 μm.

In many aspects, the dry powder formulation containing 7% by weight of the carboxamide compound of formula I comprises, consists essentially of, or consists of a fine particle mass of 128 μg for a nominal dose of carboxamide compound of formula I of 441 μg. In certain aspects, the carboxamide compound of formula I has a mean fine particle fraction measured in percent total impact recovery (TIR) from a next generation impactor (NGI) (FPF % TIR) of from about 31% to about 37%, values applicable to MGR002. Testing of the MGR002 product at 25° C. and 60% relative humidity (RH), 30° C. and 65% RH and 40° C. and 75% RH for about 6 months yielded fine particle mass (FPM) values from about 110 μg to about 160 μg. In other aspects, the dry powder formulation comprises a fine powder and the dose of the powder also known as the fine particle dose (FPD) is from about 20 μg to about 160 μg, and in yet other aspects, from about 161 μg to about 245 μg. Aerosolization performance data across the foregoing FPD range indicated fine particle fractions (FPFs) of from about 20% to about 40% or from about 25% to about 35%.

In yet other aspects, the powder formulation is dry and it includes from about 0.1, 0.2, 0.25, 0.3, 0.35, 0.4, 0.45, 0.5, 0.55, 0.6, 0.7, 0.8, 0.9, 1.0, 2.0, 3.0, 4.0, 5.0, 6.0, 7.0, 8.0, 9.0, to about 10.0% weight by weight (w/w) of carboxamide compound of formula I or carboxamide compound of formula II in lactose monohydrate. The dry powder formulation described in this application is for use in a dry powder inhaler which comprises a plurality of pockets, each pocket configured to contain the carboxamide compound at a nominal fill weight of from about 6.3 mg, 6.4 mg, 6.5 mg, 6.6 mg to about 7.0 mg. In some embodiments, each pocket is configured to contain the carboxamide compound at a nominal fill weight of from about 0.1, 0.5, 1, 2, 3, 4, 5, 6, 7, 8, 9, 10, 11, 12, 13, 14, 15, 16, 17, 18, 19, 20, 21, 22, 23, 24 to about 25 mg. A useful dry powder inhaler is known as the CRC749 inhaler and has been described in U.S. Pat. Nos. 9,399,103 and/or 9,561,336, incorporated herein by reference as if set forth in full.

In certain aspects, the powder formulation is dry and comprises: (i) from about 0.25% by weight to about 10% by weight of the carboxamide compound of formula I; or (ii) from about 0.34% by weight to about 6.00% by weight of the carboxamide compound of formula I. In other aspects, a nominal dose of carboxamide compound of formula I per nominal pocket fill weight comprises, consists essentially of, or consists of: (i) from about 0.018 mg to about 0.7 mg for a total nominal fill weight in the pocket of 7 mg; or (ii) from about 0.024 mg to about 0.42 mg for a total nominal fill weight in the pocket of 7 mg; or (iii) 0.441 mg for a total nominal fill weight in the pocket of 7 mg as set forth in Tables 1 and 2 below.

TABLE 1

| | MGR002 (Carboxamide Compound | | Nominal |
|---|---|---|---|
| | % w/w in Lactose Monohydrate) | | Pocket Fill wt |
| No salt correction | Low Dose | High Dose | (mg) |
| Formulation 1 | 0.25 | 10.00 | 7.0 |
| Formulation 2 | 0.34 | 6.00 | 7.0 |
| Formulation 3 | N/A | 7.00 | 6.3 |

TABLE 2

| | MGR002 formulation composition | | | | |
|---|---|---|---|---|---|
| | Formulation 1 | | Formulation 2 | | Formulation 3 |
| No salt | 0.25% | 10.0% | 0.34% | 6.0% | 7.0% |
| correction | w/w | w/w | w/w | w/w | w/w |
| Carboxamide | 0.018 | 0.700 | 0.024 | 0.420 | 0.441 |
| Compound | | | | | |
| (mg) | | | | | |
| Lactose | 6.983 | 6.300 | 6.976 | 6.580 | 5.859 |
| Monohydrate | | | | | |
| (mg) | | | | | |
| Nominal Fill | 7.000 | 7.000 | 7.000 | 7.000 | 6.300 |
| Wt (mg) | | | | | |

In the above tables, MGR002 represents a dry powder formulation, wherein the active ingredient is the carboxamide compound of formula I or 5-[3-(3-Hydroxyphenoxy) azetidin-1-yl]-5-methyl-2, 2-diphenylhexanamide which is not present as its salt and thus, the values in these tables do not contain any salt correction. In the formulations presented in Tables 1 and 2, the amount of lactose monohydrate can vary from about 5.86 mg to about 6.98 mg without salt correction for a total nominal fill weight in the pocket of from about 6.3 mg to about 7 mg.

In other aspects, MGR002 represents a dry powder formulation, wherein the active ingredient is the carboxamide hydrochloride salt of formula II or the 5-[3-(3-Hydroxyphenoxy) azetidin-1-yl]-5-methyl-2,2-diphenylhexanamide hydrochloride and thus, the values in Tables 3 and 4 below contain the hydrochloride salt correction. In some aspects, the powder formulation is dry and comprises: (i) from about 0.27% by weight to about 10.82% by weight of the carboxamide hydrochloride of formula II; or (ii) from about 0.37% by weight to about 6.49% by weight of the carboxamide hydrochloride of formula II. In other aspects, the carboxamide hydrochloride present in the MRG002 product comprises, consists essentially of, or consists of: (i) from about 0.019 mg to about 0.757 mg for a total nominal fill weight in the pocket of 7 mg; or (ii) 0.026 mg to about 0.454 mg nominal fill weight in the pocket of 7 mg; or (iii) 0.441 mg nominal fill weight in the pocket of 7 mg. In Tables 3 and 4, the values of the formulation components are slightly higher because they are adjusted for the higher molar mass of the carboxamide hydrochloride of formula II. In Tables 3 and 4, the amount of lactose monohydrate can vary from about 5.86 mg to about 6.98 mg for a total nominal fill weight in the pocket of from about 6.3 mg to about 7 mg taken into account the salt correction.

TABLE 3

| | MGR002 (Carboxamide Compound, | | |
|---|---|---|---|
| | as hydrochloride salt, % w/w | | Nominal |
| | in Lactose Monohydrate) | | Pocket |
| With salt | Low | High | Fill wt |
| correction | Dose | Dose | (mg) |
| Formulation 1 | 0.27 | 10.82 | 7.0 |
| Formulation 2 | 0.37 | 6.49 | 7.0 |
| Formulation 3 | N/A | 7.00 | 6.3 |

Molar Mass Carboxamide Compound 444.57
Molar Mass Carboxamide hydrochloride 481.03

TABLE 4

| | MGR002 formulation composition | | | | |
|---|---|---|---|---|---|
| | Formulation 1 | | Formulation 2 | | Formulation 3 |
| With salt | 0.25% | 10.0% | 0.34% | 6.0% | 7.0% |
| correction | w/w | w/w | w/w | w/w | w/w |
| Carboxamide | 0.019 | 0.757 | 0.026 | 0.454 | 0.441 |
| hydrochloride (mg) | | | | | |
| Lactose | 6.981 | 6.243 | 6.974 | 6.546 | 5.859 |
| Monohydrate (mg) | | | | | |
| Nominal Fill Wt | 7.000 | 7.000 | 7.000 | 7.000 | 6.300 |
| (mg) | | | | | |

Molar Mass Carboxamide Compound 444.57
Molar Mass Carboxamide hydrochloride 481.03

### Powder Formulations for Double Therapy Including a Long Acting β2 Adrenoceptor Agonist

In certain aspects, powder formulations are provided for use in a dry powder inhaler, the formulations comprising one or more pharmaceutically active ingredients selected from muscarinic M₃ receptor agonists, β2-adrenoceptor agonists and/or glucocorticoid receptor agonists and at least one excipient. In some embodiments, the powder formulations of this application comprise, consist essentially of or consist of a combination of an anticholinergic or long-acting muscarinic antagonist (LAMA), a long acting β2 adrenoceptor agonist (LABA) and at least one excipient for use in a dry powder inhaler and an excipient.

In particular, in certain applications, a LAMA compound comprises, consists essentially of or consists of the carboxamide compound of formula I

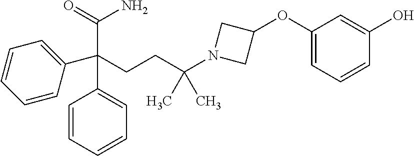 [SMILES: CC(C)(CCC(C(N)=O)(c1ccccc1)c1ccccc1)N1CC(Oc2cccc(O)c2)C1]

or a pharmaceutically acceptable salt thereof, for example a carboxamide hydrochloride of formula II

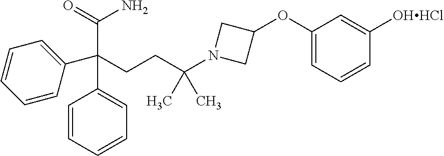

In other embodiments, a LAMA, namely the carboxamide compound of formula I can be combined with a LABA compound, for example, salmeterol as illustrated in Tables 5 and 6 below.

TABLE 5

| | FORM003A (% w/w of API for the blend added to the pocket) | | | | | | |
|---|---|---|---|---|---|---|---|
| | MGR002 Blend | | | | FORM004Blend | | Total |
| No salt | Lowest | Low | | NFW | | NFW | NFW |
| correction | dose | Dose | High Dose | (mg) | Salmeterol | (mg) | (mg) |
| Formulation 1 | 0.80 | 1.00 | 10.00 | 7.0 | 0.40 | 12.5 | 19.5 |
| Formulation 2 | N/A | N/A | 7.00 | 6.3 | 0.40 | 12.5 | 18.8 |

NFW refers to nominal fill weight of powder added to pocket

TABLE 6

| | | FORM004Aformulation composition | | | |
|---|---|---|---|---|---|
| No salt | MGR 002 | Formulation 1 | Formulation 2 | Formulation 3 | Formulation 4 |
| correction | Dose | 0.8% w/w | 1.00% w/w | 10.00% w/w | 7.0% w/w |
| Salmeterol (as | Salmeterol | 0.050 | 0.050 | 0.050 | 0.050 |
| xinafoate) (mg) | blend | | | | |
| Lactose | | 12.450 | 12.450 | 12.450 | 12.450 |
| Monohydrate | | | | | |
| (mg) | | | | | |
| Nominal Fill Wt | | 12.500 | 12.500 | 12.500 | 12.500 |
| (mg) | | | | | |
| Carboxamide | MGR002 | 0.056 | 0.070 | 0.700 | 0.441 |
| Compound (as | Blend | | | | |
| hydrochloride) | | | | | |
| (mg) | | | | | |
| Lactose | | 6.944 | 6.930 | 6.300 | 5.859 |
| Monohydrate | | | | | |
| (mg) | | | | | |
| Nominal Fill Wt | | 7.000 | 7.000 | 7.000 | 6.300 |
| (mg) | | | | | |
| Total Pocket Fill | | 19.500 | 19.500 | 19.500 | 18.800 |
| Wt (mg) | | | | | |

In Tables 5 and 6, the actives in the powder formulations are the carboxamide compound of formula I and salmeterol, both actives present not as their pharmaceutically acceptable salt, and as a result, the values of the formulation ingredients in these tables do not contain any salt correction.

In other embodiments, the powder formulation comprises, consists essentially of, or consists of LAMA and LABA compounds present as their pharmaceutically acceptable salts and at least one excipient. For example, the LAMA compound can be the carboxamide hydrochloride of formula II and the LABA compound can be salmeterol xinafoate as illustrated in Tables 7 and 8 below. The at least one excipient includes without limitations, excipients described in this application in connection with the powder formulations for monotherapy.

TABLE 7

| | FORM004A(% w/w of API for the blend added to the pocket) | | | | | | |
|---|---|---|---|---|---|---|---|
| | MGR002 (Carboxamide | | | | Salmeterol Xinafoate Blend | | |
| | hydrochloride) Blend | | | | Salmeterol | | Total |
| Salt | Lowest | Low | High | NFW | (as | NFW | NFW |
| correction | dose | Dose | Dose | (mg) | Xinafoate) | (mg) | (mg) |
| Formulation 1 | 0.87 | 1.08 | 10.82 | 7.0 | 0.58 | 12.5 | 19.5 |
| Formulation 2 | N/A | N/A | 7.57 | 6.3 | 0.58 | 12.5 | 18.8 |

NFW refers to nominal fill weight of powder added to pocket

TABLE 8

| | | FORM004formulation composition | | | |
|---|---|---|---|---|---|
| Salt correction | | Formulation 1 | Formulation 2 | Formulation 3 | Formulation 4 |
| Salmeterol (as | Salmeterol | 0.073 | 0.073 | 0.073 | 0.073 |
| xinafoate) (mg) | Xinafoate | | | | |
| Lactose | blend | 12.427 | 12.427 | 12.427 | 12.427 |
| Monohydrate | | | | | |
| (mg) | | | | | |
| Nominal Fill Wt | | 12.500 | 12.500 | 12.500 | 12.500 |
| (mg) | | | | | |
| Carboxamide | MGR002 | 0.061 | 0.076 | 0.757 | 0.477 |
| Compound (as | Blend | | | | |
| hydrochloride) | | | | | |
| (mg) | | | | | |
| Lactose | | 6.939 | 6.924 | 6.243 | 5.823 |
| Monohydrate | | | | | |
| (mg) | | | | | |
| Nominal Fill Wt | | 7.000 | 7.000 | 7.000 | 6.300 |
| (mg) | | | | | |
| Total Pocket Fill | | 19.500 | 19.500 | 19.500 | 18.800 |
| Wt (mg) | | | | | |

Molar Mass Carboxamide Compound 444.57
Molar Mass carboxamide hydrochloride 481.03
Molar Mass salmeterol 415.57
Molar Mass salmeterol xinafoate 603.75

In Tables 7 and 8, the values of the formulation components are slightly higher because they are adjusted for the higher molar mass of the carboxamide hydrochloride of formula II and the salmeterol xinafoate. In the embodiments illustrated in Tables 5 to 8, the formulation FORM004 is a combination of a carboxamide blend to be disposed in a first layer of a dry inhaler pocket and a salmeterol blend to be disposed in a second layer of the same dry inhaler pocket.

In particular, as with powder formulations containing only the carboxamide compound of formula I as found in MGR002, the powder formulations of salmeterol xinafoate also comprise pharmaceutically acceptable excipients useful for inhalable compositions. For example, MGR002 powder formulation can comprise a first excipient blended with the carboxamide compound of formula I or the carboxamide hydrochloride of formula II to form a first dry powder disposed as a first layer in a dry inhaler pocket. Salmeterol or salmeterol xinafoate comprises a second excipient which is blended to form a second dry powder disposed as a second layer in a dry inhaler pocket. Finally, the first and second excipients may comprise glucose, arabinose, lactose, sucrose, maltose, dextrans, or a combination thereof. In some embodiments, the excipient can include, but is not limited to, monosaccharides such as galactose, mannose, sorbose; disaccharides such as lactose, sucrose and trehalose and the like; polysaccharides such as starch, raffinose, dextran and the like; sugar alcohols (including glycerol, erythritol, arabitol, xylitol, sorbitol, mannitol); glycols (including ethylene glycol, propylene glycol, polyethylene glycol, polypropylene glycol); cellulose-like polymers (including hydroxy cellulose, hydroxy propyl cellulose); insoluble additives (crystalline cellulose, chitosan, calcium carbonate, talc, titanium oxide) or silica (silicon oxide), and mixtures thereof. In one aspect, the excipients present in both MGR002 and salmeterol xinafoate blend comprise, consist essentially of, or consist of lactose monohydrate. In some aspects, the powder formulation contains one, two, and/or three actives blended together into a single blend. In some embodiments, both actives, LAMA, LABA and/or the at least one excipient blended together in a mixture and added to the dry powder inhaler pocket in one layer as illustrated in Tables 9 to 13a. Tables 9-13a illustrate two blends being blended together to make one blend. However, it will be understood by those of ordinary skill in the art that all ingredients can be blended together into a single blend.

TABLE 9

| | FORM004 (% w/w of API for the blend added to the pocket) | | | | | |
|---|---|---|---|---|---|---|
| | MGR002 Blend | | | | | |
| | Lowest dose | Low Dose | High Dose | Salmeterol | MGR002: | Total |
| No salt | Carboxamide | Carboxamide | Carboxamide | Blend | Salmeterol | NFW |
| correction | Compound | Compound | Compound | Salmeterol | Blend ratio | (mg) |
| Formulation 1 | 0.80 | 1.00 | 10.00 | 0.40 | 7.0:12.5 | 19.5 |
| Formulation 2 | N/A | N/A | 7.00 | 0.40 | 6.3:12.5 | 18.8 |

NFW refers to nominal fill weight of powder added to pocket

TABLE 10

| | FORM003A(% w/w of API within the pocket, once both blends added) | | | | |
|---|---|---|---|---|---|
| | MGR002 Blend | | | | |
| | Lowest dose | Low Dose | High Dose | | Total |
| No salt | Carboxamide | Carboxamide | Carboxamide | Salmeterol Blend | NFW |
| correction | Compound | Compound | Compound | Salmeterol | (mg) |
| Formulation 1 | 0.29 | 0.36 | 3.59 | 0.26 | 19.5 |
| Formulation 2 | N/A | N/A | 2.35 | 0.27 | 18.8 |

NFW refers to nominal fill weight of powder added to pocket

TABLE 11

| | FORM003Aformulation composition | | | |
|---|---|---|---|---|
| No salt correction | Formulation 1 | Formulation 2 | Formulation 3 | Formulation 4 |
| Salmeterol (mg) | 0.050 | 0.050 | 0.050 | 0.050 |
| Carboxamide Compound (mg) | 0.056 | 0.070 | 0.700 | 0.441 |
| Lactose Monohydrate (mg) | 19.394 | 19.380 | 18.750 | 18.319 |
| Total Pocket Fill Wt (mg) | 19.500 | 19.500 | 19.500 | 18.800 |

TABLE 11a

| | | FORM003A formulation composition | | | |
|---|---|---|---|---|---|
| No salt correction | | Formulation 1 | Formulation 2 | Formulation 3 | Formulation 4 |
| Salmeterol (mg) | Salmeterol | 0.050 | 0.050 | 0.050 | 0.050 |
| Lactose Monohydrate (mg) | Blend | 12.45 | 12.45 | 12.45 | 12.45 |
| Carboxamide Compound (mg) | MGR002 | 0.056 | 0.070 | 0.700 | 0.441 |
| Lactose Monohydrate (mg) | blend | 6.944 | 6.930 | 6.300 | 5.859 |
| Total Pocket Fill Wt (mg) | | 19.500 | 19.500 | 19.500 | 18.800 |

TABLE 12

| | FORM003A (% w/w of API within the | | | | |
|---|---|---|---|---|---|
| | pocket, once both blends added) | | | | |
| | MGR002 (Carboxamide | | | | |
| | hydrochloride) Blend | | | FORM004Blend | Total |
| Salt | Lowest | Low | High | Salmeterol | NFW |
| correction | dose | Dose | Dose | (as Xinafoate) | (mg) |
| Formulation 1 | 0.31 | 0.39 | 3.88 | 0.37 | 19.5 |
| Formulation 2 | N/A | N/A | 2.54 | 0.39 | 18.8 |

NFW refers to nominal fill weight of powder added to pocket

TABLE 13

| | FORM003A formulation composition | | | |
|---|---|---|---|---|
| Salt correction | Formulation 1 | Formulation 2 | Formulation 3 | Formulation 4 |
| Salmeterol (as xinafoate)(mg) | 0.073 | 0.073 | 0.073 | 0.073 |
| Carboxamide Compound (as | 0.061 | 0.076 | 0.757 | 0.477 |
| hydrochloride)(mg) | | | | |
| Lactose Monohydrate (mg) | 19.367 | 19.352 | 18.670 | 18.250 |
| Total Pocket Fill Wt (mg) | 19.500 | 19.500 | 19.500 | 18.800 |

Molar Mass Carboxamide Compound 444.57
Molar Mass carboxamide hydrochloride 481.03
Molar Mass salmeterol 415.57
Molar Mass salmeterol xinafoate 603.75

TABLE 13a

| | | FORM003Aformulation composition | | | |
|---|---|---|---|---|---|
| No salt correction | | Formulation 1 | Formulation 2 | Formulation 3 | Formulation 4 |
| Salmeterol (mg) | Salmeterol | 0.073 | 0.073 | 0.073 | 0.073 |
| Lactose Monohydrate (mg) | Blend | 12.427 | 12.427 | 12.427 | 12.427 |
| Carboxamide Compound (mg) | MGR002 | 0.061 | 0.076 | 0.757 | 0.477 |
| Lactose Monohydrate (mg) | blend | 6.939 | 6.924 | 6.243 | 5.823 |
| Total Pocket Fill Wt (mg) | | 19.500 | 19.500 | 19.500 | 18.800 |

Molar Mass Carboxamide Compound 444.57
Molar Mass carboxamide hydrochloride 481.03
Molar Mass salmeterol 415.57
Molar Mass salmeterol xinafoate 603.75

As with the powder formulations for dual therapy, in Tables 9 to 11, the active ingredients are not in a pharmaceutically acceptable salt and thus the values of each component are not salt corrected. However, in Tables 12 and 13, the actives are present in their pharmaceutically acceptable salts, for example, MGR002 contains the carboxamide hydrochloride of formula II and the salmeterol is present as salmeterol xinafoate, and thus the values of each ingredient is salt corrected.

### Powder Formulations for Double Therapy Including an Inhalable Corticosteroid

In other embodiments, powder formulations are provided which comprise, consist essentially of, or consist of a LAMA compound, for example, the carboxamide compound of formula I or its pharmaceutically acceptable salt of carboxamide hydrochloride of formula II and as the second active an inhalable corticosteroid (ICS), for example, fluticasone propionate as illustrated in Tables 14 to 22. In Tables 14 to 17, the dry formulation FORM003B contains MRG002 present in a first layer in a dry inhaler pocket. As before, MRG002 can include a carboxamide compound of formula I and at least one excipient as in Tables 14 and 15 or a carboxamide hydrochloride of formula II and at least an excipient as in Tables 16 and 17. In Tables 14 to 17, fluticasone propionate and at least an excipient is present as FORM005 and can form a second layer in the same dry inhaler pocket.

TABLE 14

| | FORM003B(% w/w of API for the blend added to the pocket) | | | | | | |
|---|---|---|---|---|---|---|---|
| | MGR002 Blend | | | | | | |
| | Lowest dose | Low Dose | High Dose | | FORM005 Blend | | Total |
| No salt | Carboxamide | Carboxamide | Carboxamide | NFW | Fluticasone | NFW | NFW |
| correction | Compound | Compound | Compound | (mg) | Propionate | (mg) | (mg) |
| Formulation 1 | 0.80 | 1.00 | 10.00 | 7.0 | 2.00 | 12.5 | 19.5 |
| Formulation 2 | N/A | N/A | 7.00 | 6.3 | 2.00 | 12.5 | 18.8 |

NFW refers to nominal fill weight of powder added to pocket

TABLE 15

| | | FORM003B formulation composition | | | |
|---|---|---|---|---|---|
| | MGR002 | Formulation 1 | Formulation 2 | Formulation 3 | Formulation 4 |
| No salt correction | Dose | 0.8% w/w | 1.00% w/w | 10.00% w/w | 7.0% w/w |
| Fluticasone Propionate (mg) | | 0.250 | | | |
| Lactose Monohydrate (mg) | | 12.250 | | | |
| Nominal Fill Wt (mg) | | 12.500 | | | |
| Carboxamide Compound (mg) | MGR002 | 0.056 | | | |
| Lactose Monohydrate (mg) | Blend | 6.944 | 0.070 | 0.700 | 0.441 |
| Nominal Fill Wt (mg) | | 7.000 | 6.930 | 6.300 | 5.859 |
| Total Pocket Fill Wt (mg) | | 19.500 | 7.000 | 7.000 | 6.300 |

NFW refers to nominal fill weight of powder added to pocket

TABLE 16

| | FORM003B (% w/w of API for the blend added to the pocket) | | | | | | |
|---|---|---|---|---|---|---|---|
| | MGR002 (Carboxamide | | | | | | |
| | hydrochloride) Blend | | | | FORM005 Blend | | Total |
| Salt | Lowest | Low | High | NFW | Fluticasone | NFW | NFW |
| correction | dose | Dose | Dose | (mg) | Propionate | (mg) | (mg) |
| Formulation 1 | 0.87 | 1.08 | 10.82 | 7.0 | 2.00 | 12.5 | 19.5 |
| Formulation 2 | N/A | N/A | 7.57 | 6.3 | 2.00 | 12.5 | 18.8 |

NFW refers to nominal fill weight of powder added to pocket

TABLE 17

| | | FORM003B formulation composition | | | |
|---|---|---|---|---|---|
| | MGR002 | Formulation 1 | Formulation 2 | Formulation 3 | Formulation 4 |
| Salt correction | Dose | 0.8% w/w | 1.00% w/w | 10.00% w/w | 7.0% w/w |
| Fluticasone Propionate (mg) | | 0.250 | | | |
| Lactose Monohydrate (mg) | | 12.252 | | | |
| Nominal Fill Wt (mg) | | 12.500 | | | |
| Carboxamide Compound (as | MGR002 | 0.061 | 0.076 | 0.757 | 0.477 |
| hydrochloride)(mg) | Blend | | | | |
| Lactose Monohydrate (mg) | | 6.939 | 6.924 | 6.243 | 5.823 |
| Nominal Fill Wt (mg) | | 7.000 | 7.000 | 7.000 | 6.300 |
| Total Pocket Fill Wt (mg) | | 19.500 | 19.500 | 19.500 | 18.800 |

As with powder formulations containing a LABA compound in combination with the carboxamide compound of formula I or II, the powder formulations containing an ICS are in some cases salt adjusted and in other cases not salt adjusted. For example, in Tables 14 and 15, the values of the carboxamide compound of formula I is not salt adjusted because it is not used as its pharmaceutically acceptable hydrochloride salt. On the other hand, in Tables 16 and 17, the values of the carboxamide hydrochloride component are slightly higher as they have been salt corrected.

As with other powder formulations, powder formulations comprising LAMA/ICS compounds and at least one excipient can be added to a dry powder inhaler pocket blended together in a mixture, and not disposed into layers, as illustrated in Tables 18 to 22.

TABLE 18

| | FORM003B (% w/w of API for the blend added to the pocket) | | | | | | |
|---|---|---|---|---|---|---|---|
| | MGR002 Blend | | | | | | |
| | Lowest dose | Low Dose | High Dose | | FORM005 Blend | | Total |
| No salt | Carboxamide | Carboxamide | Carboxamide | NFW | Fluticasone | NFW | NFW |
| correction | Compound | Compound | Compound | (mg) | Propionate | (mg) | (mg) |
| Formulation 1 | 0.80 | 1.00 | 10.00 | 7.0 | 2.00 | 12.5 | 19.5 |
| Formulation 2 | N/A | N/A | 7.00 | 6.3 | 2.00 | 12.5 | 18.8 |

NFW refers to nominal fill weight of powder added to pocket

TABLE 19

| | FORM003B (% w/w of API within the pocket, once both blends added) | | | | |
|---|---|---|---|---|---|
| | MGR002 Blend | | | | |
| | Lowest dose | Low Dose | High Dose | FORM005 Blend | Total |
| No salt | Carboxamide | Carboxamide | Carboxamide | Fluticasone | NFW |
| correction | Compound | Compound | Compound | Propionate | (mg) |
| Formulation 1 | 0.29 | 0.36 | 3.59 | 1.28 | 19.5 |
| Formulation 2 | N/A | N/A | 2.35 | 1.33 | 18.8 |

NFW refers to nominal fill weight of powder added to pocket

TABLE 20

| | | FORM003B formulation composition | | | |
|---|---|---|---|---|---|
| | MGR002 | Formulation 1 | Formulation 2 | Formulation 3 | Formulation 4 |
| No salt correction | Dose | 0.8% w/w | 1.00% w/w | 10.00% w/w | 7.0% w/w |
| Fluticasone Propionate (mg) | | 0.250 | | | 0.250 |
| Carboxamide Compound (mg) | MGR002 | 0.056 | 0.070 | 0.700 | 0.441 |
| Lactose Monohydrate (mg) | Blend | 19.194 | 19.18 | 18.55 | 18.11 |
| Total Pocket Fill Wt (mg) | | 19.500 | 19.500 | 19.500 | 18.800 |

As with other powder formulations, the values of the components in Tables 18 to 20 are based on the carboxamide compound of formula I and thus they do not contain any salt correction. By contrast, in Tables 21 and 22, the carboxamide compound is present as its hydrochloride salt and thus, the values set forth in these tables include a salt correction.

TABLE 21

| | FORM003B (% w/w of API within the | | | | |
|---|---|---|---|---|---|
| | pocket, once both blends added) | | | | |
| | MGR002 (Carboxamide | | | FORM005 | |
| | hydrochloride) Blend | | | Blend | Total |
| Salt | Lowest | Low | High | Fluticasone | NFW |
| correction | dose | Dose | Dose | Propionate | (mg) |
| Formulation 1 | 0.31 | 0.39 | 3.88 | 1.28 | 19.5 |
| Formulation 2 | N/A | N/A | 2.54 | 1.33 | 18.8 |

NFW refers to nominal fill weight of powder added to pocket

TABLE 22

| | | FORM003Bformulation composition | | | |
|---|---|---|---|---|---|
| | MGR002 | Formulation 1 | Formulation 2 | Formulation 3 | Formulation 4 |
| Salt correction | Dose | 0.8% w/w | 1.00% w/w | 10.00% w/w | 7.0% w/w |
| Fluticasone Propionate (mg) | | 0.250 | | | 0.250 |
| Carboxamide Compound (as | MGR002 | 0.061 | 0.076 | 0.757 | 0.477 |
| hydrochloride)(mg) | | | | | |
| Lactose Monohydrate (mg) | | 19.19 | 19.175 | 18.493 | 18.073 |
| Total Pocket Fill Wt (mg) | | 19.500 | 19.500 | 19.500 | 18.800 |

Molar Mass Carboxamide Compound 444.57
Molar Mass carboxamide hydrochloride 481.03
Molar Mass salmeterol 415.57
Molar Mass salmeterol xinafoate 603.75

### Powder Formulations for Triple Therapy

In certain embodiments, powder formulations are provided which comprise, consist essentially of, or consist of a combination of an anticholinergic or long-acting muscarinic antagonist (LAMA), a long acting β2 adrenoceptor agonist (LABA), an inhalable corticosteroid (ICS) and at least one excipient. These powder formulations are provided for use in a dry powder inhaler having pockets to be disposed in separate layers in each pocket or as a mixture. In particular, in some applications, the triple therapy powder formulations can include in a first layer, disposed in a dry inhaler pocket, a LAMA compound with or without at least one excipient; in a second layer, superimposed upon the first layer, a LABA compound with or without at least one excipient; and in a third layer, superimposed upon the second layer, an ICS compound with or without at least an excipient. In other aspects, a dry powder formulation for triple therapy can be used in a dry powder inhaler which comprises a plurality of pockets, each pocket configured to comprise, consist essentially of or consist of at least two blend layers, each layer comprising a first blend containing a carboxamide compound of formula I or II and at least one excipient and a second blend containing fluticasone propionate and salmeterol or salmeterol xinafoate.

In certain aspects, a LAMA compound comprises, consists essentially of, or consists of the carboxamide compound of formula I

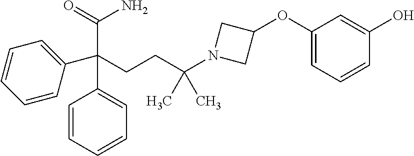 [SMILES: CC(C)(CCC(C(N)=O)(c1ccccc1)c1ccccc1)N1CC(Oc2cccc(O)c2)C1]

or a pharmaceutically acceptable salt thereof, for example, a carboxamide hydrochloride of formula II

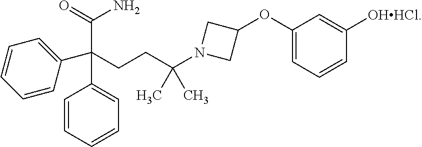

The long acting β2 adrenoceptor agonist comprises, consists essentially of, or consists of salmeterol or salmeterol xinafoate and the inhalable corticosteroid comprises, consists essentially of, or consists of fluticasone propionate. The carboxamide compound of formula I is also known as 5-[3-(3-Hydroxyphenoxy)azetidin-1-yl]-5-methyl-2,2-diphenylhexanamide. A formulation which includes three different active ingredients, such as a carboxamide compound of formula I or II, salmeterol or salmeterol xinafoate, fluticasone propionate with or without an excipient, for example, lactose monohydrate, would be beneficial to provide a dry powder inhalation product for use as triple therapy to treat patients who could benefit from the different therapeutic properties of each active ingredient.

In some embodiments, the carboxamide compound of formula I or the 5-[3-(3-Hydroxyphenoxy)azetidin-1-yl]-5-methyl-2,2-diphenylhexanamide hydrochloride of formula II in the triple therapy powder formulation is in a dose of from about 40 to about 800 μg, the salmeterol xinafoate is in a dose of from about 72.5 μg, and the fluticasone propionate is in a dose of from about 100 to about 500 μg.

In the triple therapy powder formulations, the carboxamide compound of formula I or 5-[3-(3-Hydroxyphenoxy)azetidin-1-yl]-5-methyl-2,2-diphenylhexanamide hydrochloride of formula II is in a dose of from about 40 to about 800 μg. In some applications, the 5-[3-(3-Hydroxyphenoxy)azetidin-1-yl]-5-methyl-2,2-diphenylhexanamide hydrochloride is in a dose of from about 40 to about 700 μg, from about 40 to about 600 μg, from about 40 to about 500 μg, from about 40 to about 400 μg, from about 40 to about 300 μg, from about 40 to about 200 μg, from about 40 to about 100 μg, from about 100 to about 800 μg, from about 100 to about 700 μg, from about 100 to about 600 μg, from about 100 to about 500 μg, from about 100 to about 400 μg, from about 100 to about 300 μg, from about 100 to about 200 μg, or from about 100 to about 150 μg.

In certain embodiments, the carboxamide compound of formula I or 5-[3-(3-Hydroxyphenoxy)azetidin-1-yl]-5-methyl-2,2-diphenylhexanamide hydrochloride of formula II is in a dose of from about 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690 to about 700 μg.

In other embodiments, the carboxamide compound of formula I or 5-[3-(3-Hydroxyphenoxy)azetidin-1-yl]-5-methyl-2,2-diphenylhexanamide hydrochloride of formula II is in an amount from about 0.01 to about 99 wt. %. In some embodiments, the -[3-(3-Hydroxyphenoxy)azetidin-1-yl]-5-methyl-2,2-diphenyl-hexanamide hydrochloride is in an amount about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84 85, 86, 87, 88, 89, 90, 91, 92, 93, 94, 95, 96, 97, 98 or 99 wt % of the triple therapy powder formulation.

The salmeterol is in a dose of about 50 μg or salmeterol xinafoate is in a dose of about 72.5 μg. In some embodiments, in the triple therapy powder formulation, the salmeterol or salmeterol xinafoate is in a dose of from about 1, 2, 3, 4, or 5 μg to about 100 μg, from about 5 to about 75 μg, from about 10 to about 100 μg, from about 10 to about 75 μg, from about 10 to about 50 μg, from about 25 to about 100 μg, from about 25 to about 75 μg, or from about 25 to about 50 μg.

In other embodiments, in the triple therapy powder formulations, the salmeterol or salmeterol xinafoate is in a dose of about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 to about 100 μg of the formulation.

In some embodiments, the salmeterol xinafoate is in an amount of about 0.01 to about 99 wt. % of the triple therapy powder formulation. In some embodiments, the salmeterol or salmeterol xinafoate is in an amount about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84 85, 86, 87, 88, 89, 90, 91, 92, 93, 94, 95, 96, 97, 98 or 99 wt % of the triple therapy powder formulation.

In some embodiments, the fluticasone propionate is in a dose of about 50, 60, 70, 80, 90, 100 to about 500 μg. In some embodiments, the fluticasone propionate is in a dose of about 100 to about 400 μg, about 100 to about 300 μg, about 100 to about 200 μg, about 100 to about 150 μg, about 200 to about 500 μg, about 200 to about 400 μg, about 200 to about 300 μg, about 300 to about 500 μg, or about 300 to about 400 μg of in the triple therapy powder formulation.

In some embodiments, the fluticasone propionate is in a dose of from about 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490 to about 500 μg of the triple therapy powder formulation.

In some embodiments, the fluticasone propionate is in an amount of from about 0.01 to about 99 wt. % of the triple therapy powder formulation. In some embodiments, the fluticasone propionate is in an amount about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84 85, 86, 87, 88, 89, 90, 91, 92, 93, 94, 95, 96, 97, 98 or 99 wt % of the first and/or the second layer.

In the triple therapy powder formulation, the carboxamide compound of formula I or 5-[3-(3-Hydroxyphenoxy)azetidin-1-yl]-5-methyl-2,2-diphenylhexanamide hydrochloride of formula II has a particle size of from about less than 10 μm to about less than 5 μm, less than 4 μm, less than 3 μm, less than 2 μm, less than 1 μm. In some embodiments, the carboxamide compound of formula I or 5-[3-(3-Hydroxyphenoxy)azetidin-1-yl]-5-methyl-2,2-diphenylhexanamide hydrochloride of formula II has a particle size of from about less than 1 μm, less than 2 μm, less than 3 μm, less than 4 μm, less than 5 μm, less than 6 μm, less than 7 μm, less than 8 μm, less than 9 μm, to about less than 10 μm.

In the triple therapy powder formulation, the salmeterol or salmeterol xinafoate has a particle size of from about less than 10 μm to about less than 5 μm, less than 4 μm, less than 3 μm, less than 2 μm, less than 1 μm. In some embodiments, the salmeterol or salmeterol xinafoate has a particle size of from about less than 5 μm, less than 6 μm, less than 7 μm, less than 8 μm, less than 9 μm, to about less than 10 μm.

In the triple therapy powder formulation, the fluticasone propionate has a particle size from about less than 10 μm to about less than 5 μm, less than 4 μm, less than 3 μm, less than 2 μm, less than 1 μm. In some embodiments, the fluticasone propionate has a particle size of from about less than 1 μm, less than 2 μm, less than 3 μm, less than 4 μm, less than 5 μm, less than 6 μm, less than 7 μm, less than 8 μm, less than 9 μm, to about less than 10 μm.

In some embodiments, the excipient can include, but is not limited to, monosaccharides such as galactose, mannose, sorbose; disaccharides such as lactose, sucrose and trehalose and the like; polysaccharides such as starch, raffinose, dextran and the like; and mixtures thereof. In some embodiments, the lactose is lactose monohydrate. The lactose can include, but is not limited to, Lactohale LH200 having D₅₀ of 60 μm or Lactohale LH200 having D₅₀ of 80 μm.

In some embodiments, the excipient comprises from about 0.01 to about 99.9 wt. % of the triple therapy powder formulation. In some embodiments, the excipient comprises, consists essentially of, or consists of from about 0.1 to about 99 wt. %, from about 1 to about 99 wt. %, from about 10 to about 99 wt. %, from about 20 to about 99 wt. %, from about 30 to about 99 wt. %, from about 40 to about 99 wt. %, from about 50 to about 99 wt. %, from about 60 to about 99 wt. %, from about 70 to about 99 wt. %, from about 80 to about 99 wt. %, from about 90 to about 99 wt. %, from about 95 to about 99 wt. %, or from about 97 to about 99 wt. % of the triple therapy powder formulation.

In some embodiments, the excipient comprises, consists essentially of, or consists of about 0.1, 0.2, 0.3, 0.4, 0.5, 0.6, 0.7, 0.8, 0.9, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 99.9 wt. % of the triple therapy powder formulation.

In some embodiments, the excipient is a powder and has an average particle size of from about 1, 2, 3, 4, 5, 6, 7, 8, 9, 10 to about 200 μm. In some embodiments, the excipient has an average particle size of from about 10 to about 150 μm, from about 10 to about 100 μm, from about 10 to about 75 μm, from about 10 to about 50 μm, from about 25 to about from about 150 μm, from about 25 to about 100 μm, from about 25 to about 75 μm, or from about 25 to about 50 μm. In some embodiments, the excipient has an average particle size of from about 10, 20, 30, 40, 50, 60, 70, 80, 90, 100, 110, 120, 130, 140, 150, 160, 170, 180, 190 to about 200 μm. The excipient can be a coarse powder or a fine powder or a mixture thereof.

In various embodiments, powder formulations comprising three different active ingredients include from about 0.8%, 0.9%, 1.0%, 2.0%, 3.0%, 4.0%, 5.0%, 6.0%, 7.0%, 8.0%, 9.0% to about 10.0% weight by weight (w/w) of carboxamide compound of formula I in lactose monohydrate. A dry powder formulation for triple therapy can be used in a dry powder inhaler which comprises a plurality of pockets, each pocket configured to comprise, consist essentially of, or consist of at least two blend layers, each layer comprising a first blend containing from about 0.8% to about 10% by weight of the carboxamide compound of formula I filled to a nominal weight of about 7 mg in the pocket and a second blend containing about 2% by weight of fluticasone propionate and 0.4% by weight salmeterol filled to a nominal 12.5 mg in the pocket, both blends filled to a total nominal pocket fill weight of from about 18.8 mg to about 19.5 mg as illustrated in Table 23 below.

TABLE 23

| | MGR003 (% w/w of API for the blend added to the pocket) | | | | | | | |
|---|---|---|---|---|---|---|---|---|
| | MGR002 Blend | | | | | | | |
| | Lowest dose | Low Dose | High Dose | | MGR001 Blend | | | Total |
| No salt | Carboxamide | Carboxamide | Carboxamide | NFW | Fluticasone | | NFW | NFW |
| correction | Compound | Compound | Compound | (mg) | Propionate | Salmeterol | (mg) | (mg) |
| Formulation 1 | 0.80 | 1.00 | 10.00 | 7.0 | 2.00 | 0.40 | 12.5 | 19.5 |
| Formulation 2 | N/A | N/A | 7.00 | 6.3 | 2.00 | 0.40 | 12.5 | 18.8 |

NFW refers nominal fill weight of powder added to pocket

In another embodiment, the first blend contains from about 0.056 mg to about 0.441 mg of the carboxamide compound of formula I and from about 5.86 mg to about 6.94 mg of lactose monohydrate filled to a nominal fill weight of from about 6.3 mg to about 7.0 mg, and a second blend containing from about 0.05 mg salmeterol, about 0.250 mg of fluticasone propionate and 12.2 mg lactose monohydrate, both blends filled to a total pocket fill weight of from about 18.8 mg to about 19.5 mg for a total pocket fill weight of from about 18.8 mg to about 19.5 mg as illustrated in Table 24 below. In a certain embodiment, the first blend contains from about 0.87 mg to about 10.82 mg of the carboxamide compound of formula II filled to a nominal 7 mg of powder added to the pocket, and a second blend containing from about 2.00 mg of fluticasone propionate, from about 0.58 of salmeterol xinafoate, both blends filled to a total nominal fill weight of from about 18.8 to about 19.5 as illustrated in Table 25 below. In another embodiment, the first blend contains from about 0.061 mg to about 0.477 mg of the carboxamide compound of formula II and from about 5.823 mg to about 6.939 mg of lactose mononhydrate for a nominal fill weight of 7.0 mg, and a second blend from about 0.25 mg of fluticasone propionate and about 0.73 mg of salmeterol xinafoate and 12.177 mg lactose monohydrate for a nominal fill weight of 12.5 mg, both blends filled to a total nominal fill weight of from about 18.8 to about 19.5 as illustrated in table 26 below.

TABLE 24

| | | MGR003 formulation composition | | | |
|---|---|---|---|---|---|
| | | Formulation 1 | Formulation 2 | Formulation 3 | Formulation 4 |
| No salt correction | 002 Dose | 0.8% w/w | 1.00% w/w | 10.00% w/w | 7.0% w/w |
| Fluticasone Propionate | MGR001 | 0.250 | | | |
| (mg) | Blend | | | | |
| Salmeterol (mg) | | 0.050 | | | |
| Lactose Monohydrate (mg) | | 12.200 | | | |
| Nominal Fill Wt (mg) | | 12.500 | | | |
| Carboxamide Compound | MGR002 | 0.056 | 0.070 | 0.700 | 0.441 |
| (mg) | Blend | | | | |
| Lactose Monohydrate | | 6.944 | 6.930 | 6.300 | 5.859 |
| (mg) | | | | | |
| Nominal Fill Wt (mg) | | 7.000 | 7.000 | 7.000 | 6.300 |
| Total Pocket Fill Wt (mg) | | 19.500 | 19.500 | 19.500 | 18.800 |

In Tables 23 and 24, the compounds present in the blends of MGR001 and MGR002 are not present as their salts and thus, the values in these tables do not contain a salt correction. In the formulations presented in Table 24, the amount of lactose monohydrate can vary from about 5.86 mg in MGR002 to about 12.2 mg in MGR001, again without a salt correction.

In certain applications, the MGR002 blend used in the preparation of MGR003 ranges from about 0.8 to about 10.0% w/w of the carboxamide compound of formula I present in lactose monohydrate, which is filled to a nominal 7 mg in the inhaler pocket with no correction for the hydrochloride salt form used as illustrated in Tables 23 and 24. Additionally, MGR001 has about 2% w/w fluticasone propionate and about 0.4% w/w salmeterol in lactose monohydrate, which is filled to a nominal weight of 12.5 mg in the inhaler pocket with no correction for the xinafoate salt form of salmeterol, both used for a total nominal pocket fill weight of 19.5 mg.

In other aspects, MGR002 represents a dry powder formulation, wherein the active ingredient is the hydrochloride salt of the carboxamide compound 5-[3-(3-Hydroxyphenoxy) azetidin-1-yl]-5-methyl-2,2-diphenylhexanamide. Further, the salmeterol of MGR001 is present as the xinafoate salt and thus, the values in Tables 25 and 26 below contain a salt correction. In table 26, the amount of lactose monohydrate can vary from about 5.82 mg in MGR002 to about 12.18 mg in MGR001 taken into account the salt correction.

TABLE 25

| | MGR003 (% w/w of API for the blend added to the pocket) | | | | | | | |
|---|---|---|---|---|---|---|---|---|
| | MGR002 (Carboxamide | | | | MGR001 Blend | | | |
| | hydrochloride) Blend | | | | | Salmeterol | | Total |
| Salt | Lowest | Low | High | NFW | Fluticasone | (as | NFW | NFW |
| correction | dose | Dose | Dose | (mg) | Propionate | Xinafoate) | (mg) | (mg) |
| Formulation 1 | 0.87 | 1.08 | 10.82 | 7.0 | 2.00 | 0.58 | 12.5 | 19.5 |
| Formulation 2 | N/A | N/A | 7.57 | 6.3 | 2.00 | 0.58 | 12.5 | 18.8 |

NFW refers to nominal fill weight of powder added to pocket

TABLE 26

| | | MGR003 formulation composition | | | |
|---|---|---|---|---|---|
| | | Formulation 1 | Formulation 2 | Formulation 3 | Formulation 4 |
| Salt correction | 002 Dose | 0.8% w/w | 1.00% w/w | 10.00% w/w | 7.0% w/w |
| Fluticasone Propionate (mg) | MGR001 | 0.250 | | | |
| Salmeterol (as xinafoate)(mg) | Blend | 0.073 | | | |
| Lactose Monohydrate (mg) | | 12.177 | | | |
| Nominal Fill Wt (mg) | | 12.500 | | | |
| Carboxamide Compound (as | MGR002 | 0.061 | 0.076 | 0.757 | 0.477 |
| hydrochloride)(mg) | Blend | | | | |
| Lactose Monohydrate (mg) | | 6.939 | 6.924 | 6.243 | 5.823 |
| Nominal Fill Wt (mg) | | 7.000 | 7.000 | 7.000 | 6.300 |
| Total Pocket Fill Wt (mg) | | 19.500 | 19.500 | 19.500 | 18.800 |

Molar Mass Carboxamide Compound 444.57
Molar Mass Carboxamide HCl 481.03
Molar Mass salmeterol 415.57
Molar Mass salmeterol xinafoate 603.75

In the above Tables 25 and 26, MGR001 represents a dry powder for inhalation in a CRC749 dry powder inhaler device of fluticasone propionate and salmeterol xinafoate in a ratio of 250 μg/50 μg. Further, MGR002 represents a dry powder formulation for inhalation (441 μg) in a CRC749 device, wherein the active ingredient is the carboxamide compound 5-[3-(3-Hydroxyphenoxy) azetidin-1-yl]-5-methyl-2,2-diphenylhexanamide hydrochloride. In addition, MGR003 refers to dry powder for inhalation in a CRC749 device a fluticasone propionate, salmeterol xinafoate and carboxamide compound of formula I as hydrochloride in a ratio of 250 μg/50 μg/441 μg.

As expected, in Tables 25 and 26 above, the dosage of the carboxamide hydrochloride and the salmeterol xinafoate are somewhat higher due to the salt correction. Since the nominal fill weight and the total pocket fill weight remain unchanged at 12.5 mg and 19.5 mg, respectively, the amount of lactose monohydrate was adjusted lower.

In other embodiments illustrated in Tables 27 to 31 below, the blends of MGR002 and MGR001 form a mixture and are disposed in the pocket of a dry inhaler as a mixture and not in separate layers. As a result, the specific mass of the carboxamide compound of formula I or formula II, fluticasone propionate, salmeterol and lactose monohydrate is the same as shown in Tables 23 to 26, however, the concentration of each active ingredient or drug within each pocket was reduced because of the specific mass of all materials added in each pocket. This is applicable to dry powder formulations with or without salt corrections.

In particular, the dry powder formulation for triple therapy can be used in a dry powder inhaler which comprises a plurality of pockets, each pocket configured to comprise at least one blend layer, each layer comprises, consists essentially of, or consists of a mixture of a first blend containing from about 0.8% to about 10% by weight of the carboxamide compound of formula I filled to a nominal weight of about 7 mg in the pocket and a second blend containing about 2% by weight of fluticasone propionate and 0.4% by weight of salmeterol filled to a nominal fill weight of 12.5 mg in the pocket as illustrated in Table 27 below. In another aspect, the mixture comprises, consists essentially of, or consists of a first blend of from about 0.29 mg to about 3.59 mg of the carboxamide compound of formula I and a second blend containing about 0.26 mg of salmeterol and from about 1.28 mg to about 1.33 mg and fluticasone propionate for a total nominal fill weight in the pocket from about 18.8 mg to about 19.5 mg as illustrated in Table 28 below. In a certain aspect, the mixture comprises, consists essentially of, or consists of from about 0.056 mg to about 0.441 mg of carboxamide compound of formula I, from about 0.250 mg of fluticasone propionate, about 0.05 mg salmeterol and from about 18.06 mg to about 19.144 mg of lactose monohydrate for a total fill weight in the pocket of from about 18.8 mg to about 19.5 mg as illustrated in Table 29 below. In yet another aspect, the mixture comprises, consists essentially of or consist of a first blend containing from about 0.31 mg to about 3.88 mg of carboxamide compound of formula II, and a second blend containing from about 1.28 mg to about 1.33 mg of fluticasone propionate, from about 0.37 mg to about 0.39 mg of salmeterol xinafoate for a total nominal fill weight in the pocket of from about 18.8 mg to about 19.5 mg as illustrated in Table 30 below. In another embodiment, the mixture comprises, consists essentially of, or consists of from about 0.061 mg to about 0.757 mg of carboxamide compound of formula II, about 0.250 fluticasone propionate and about 0.73 mg of salmeterol xinafoate and form about 18.42 mg to about 19.12 mg of lactose monohydrate for a total fill weight in the pocket of from about 18.8 to about 19.5 mg as illustrated in Table 31 below.

TABLE 27

| | MGR003 (% w/w of API for the blend added to the pocket) | | | | | | | |
|---|---|---|---|---|---|---|---|---|
| | MGR002 Blend | | | | | | | |
| | Lowest dose | Low Dose | High Dose | | MGR001 Blend | | | Total |
| No salt | Carboxamide | Carboxamide | Carboxamide | NFW | Fluticasone | | NFW | NFW |
| correction | Compound | Compound | Compound | (mg) | Propionate | Salmeterol | (mg) | (mg) |
| Formulation 1 | 0.80 | 1.00 | 10.00 | 7.0 | 2.00 | 0.40 | 12.5 | 19.5 |
| Formulation 2 | N/A | N/A | 7.00 | 6.3 | 2.00 | 0.40 | 12.5 | 18.8 |

NFW refers to nominal fill weight of powder added to pocket

TABLE 28

| | MGR003 (% w/w of API within the pocket, once both blends added) | | | | | |
|---|---|---|---|---|---|---|
| | MGR002 Blend | | | | | |
| | Lowest dose | Low Dose | High Dose | MGR001 Blend | | Total |
| No salt | Carboxamide | Carboxamide | Carboxamide | Fluticasone | | NFW |
| correction | Compound | Compound | Compound | Propionate | Salmeterol | (mg) |
| Formulation 1 | 0.29 | 0.36 | 3.59 | 1.28 | 0.26 | 19.5 |
| Formulation 2 | N/A | N/A | 2.35 | 1.33 | 0.27 | 18.8 |

NFW refers to nominal fill weight of powder added to pocket

TABLE 29

| | | MGR003 formulation composition | | | |
|---|---|---|---|---|---|
| | | Formulation 1 | Formulation 2 | Formulation 3 | Formulation 4 |
| No salt correction | 002 Dose | 0.8% w/w | 1.00% w/w | 10.00% w/w | 7.0% w/w |
| Fluticasone Propionate (mg) | MGR001 | 0.250 | | | 0.250 |
| | Blend | | | | |
| Salmeterol (mg) | MGR001 | 0.050 | | | 0.050 |
| Carboxamide Compound | MGR002 | 0.056 | 0.070 | 0.700 | 0.441 |
| (mg) | Blend | | | | |
| Lactose Monohydrate (mg) | | 19.144 | 19.130 | 18.500 | 18.059 |
| Total Pocket Fill Wt (mg) | | 19.500 | 19.500 | 19.500 | 18.800 |

TABLE 29a

| | | MGR003 formulation composition | | | |
|---|---|---|---|---|---|
| | | Formulation 1 | Formulation 2 | Formulation 3 | Formulation 4 |
| No salt correction | 002 Dose | 0.8% w/w | 1.00% w/w | 10.00% w/w | 7.0% w/w |
| Fluticasone Propionate (mg) | MGR001 | 0.250 | 0.250 | 0.250 | 0.250 |
| Salmeterol (mg) | Blend | 0.050 | 0.050 | 0.050 | 0.050 |
| Lactose Monohydrate (mg) | | 12.200 | 12.200 | 12.200 | 12.200 |
| Carboxamide Compound | MGR002 | 0.056 | 0.070 | 0.700 | 0.441 |
| (mg) | Blend | | | | |
| Lactose Monohydrate (mg) | | 6.944 | 6.930 | 6.300 | 5.859 |
| Total Pocket Fill Wt (mg) | | 19.500 | 19.500 | 19.500 | 18.800 |

TABLE 30

| | MGR003 (% w/w of API within the pocket, once both blends added) | | | | | |
|---|---|---|---|---|---|---|
| | MGR002 (Carboxamide | | | MGR001 Blend | | |
| | hydrochloride) Blend | | | | Salmeterol | Total |
| Salt | Lowest | Low | High | Fluticasone | (as | NFW |
| correction | dose | Dose | Dose | Propionate | Xinafoate) | (mg) |
| Formulation 1 | 0.31 | 0.39 | 3.88 | 1.28 | 0.37 | 19.5 |
| Formulation 2 | N/A | N/A | 2.54 | 1.33 | 0.39 | 18.8 |

NFW refers to nominal fill weight of powder added to pocket

TABLE 31

| | | MGR003 formulation composition | | | |
|---|---|---|---|---|---|
| | | Formulation 1 | Formulation 2 | Formulation 3 | Formulation 4 |
| Salt correction | 002 Dose | 0.8% w/w | 1.00% w/w | 10.00% w/w | 7.0% w/w |
| Fluticasone Propionate (mg) | MGR001 | 0.250 | | | 0.250 |
| | Blend | | | | |
| Salmeterol (as xinafoate)(mg) | MGR001 | 0.073 | | | 0.073 |
| Carboxamide Compound (as | MGR002 | 0.061 | 0.076 | 0.757 | 0.477 |
| hydrochloride)(mg) | | | | | |
| Lactose Monohydrate (mg) | | 19.117 | 19.102 | 18.420 | 18.000 |
| Total Pocket Fill Wt (mg) | | 19.500 | 19.500 | 19.500 | 18.800 |

Molar Mass Carboxamide Compound 444.57
Molar Mass carboxamide hydrochloride 481.03
Molar Mass salmeterol 415.57
Molar Mass salmeterol xinafoate 603.75

TABLE 31a

| | | MGR003 formulation composition | | | |
|---|---|---|---|---|---|
| | | Formulation 1 | Formulation 2 | Formulation 3 | Formulation 4 |
| Saltcorrection | 002 Dose | 0.8% w/w | 1.00% w/w | 10.00% w/w | 7.0% w/w |
| Fluticasone Propionate (mg) | MGR001 | 0.250 | 0.250 | 0.250 | 0.250 |
| Salmeterol (mg) | Blend | 0.073 | 0.073 | 0.073 | 0.073 |
| Lactose Monohydrate (mg) | | 12.177 | 12.177 | 12.177 | 12.177 |
| Carboxamide Compound | MGR002 | 0.061 | 0.076 | 0.757 | 0.477 |
| (mg) | Blend | | | | |
| Lactose Monohydrate (mg) | | 6.939 | 6.924 | 6.243 | 5.823 |
| Total Pocket Fill Wt (mg) | | 19.500 | 19.500 | 19.500 | 18.800 |

Molar Mass Carboxamide Compound 444.57
Molar Mass carboxamide hydrochloride 481.03
Molar Mass salmeterol 415.57
Molar Mass salmeterol xinafoate 603.75

The powder formulations containing three active ingredients including the carboxamide compound of formula II are used for inhalation in a CRC749 device wherein each formulation MGR002 and MGR001 is arranged within the device. In some embodiments, an inhalation powder formulation is provided that has a multi-layered filling to create a combination layered dry powder product.

In the powder formulations of this application, the 5-[3-(3-Hydroxyphenoxy)azetidin-1-yl]-5-methyl-2,2-diphenylhexanamide by itself or as the hydrochloride salt has a particle size of from about less than 10 μm to about less than 5 μm. In some embodiments, the 5-[3-(3-Hydroxyphenoxy)azetidin-1-yl]-5-methyl-2,2-diphenylhexanamide hydrochloride has a particle size of from about less than 1 μm, less than 2 μm, less than 3 μm, less than 4 μm, 5 μm, less than 6 μm, less than 7 μm, less than 8 μm, less than 9 μm, to about less than 10 μm.

The salmeterol xinafoate has a particle size of from about less than 10 μm to about less than 5 μm, less than 4 μm, less than 3 μm, less than 2 μm, less than 1 μm. In some embodiments, the salmeterol xinafoate has a particle size of from about less than 5 μm, less than 6 μm, less than 7 μm, less than 8 μm, less than 9 μm, to about less than 10 μm.

The fluticasone propionate has a particle size from about less than 10 μm to about less than 5 μm. In some embodiments, the fluticasone propionate has a particle size of from about less than 1 μm, less than 2 μm, less than 3 μm, less than 4 μm, 5 μm, less than 6 μm, less than 7 μm, less than 8 μm, less than 9 μm, to about less than 10 μm.

As with powder formulations containing only the carboxamide compound of formula II as found in MGR002, the powder formulations of MGR001 also comprise pharmaceutically acceptable excipients useful for inhalable compositions. For example, MGR002 powder formulation can comprise a first excipient blended with the carboxamide compound of formula II to form a first dry powder disposed as a first layer in an inhaler pocket. MGR001 which includes fluticasone propionate and salmeterol or salmeterol xinafoate comprises a second excipient which is blended to form a second dry powder disposed as a second layer in an inhaler pocket. Finally, the first and second excipients may comprise glucose, arabinose, lactose, sucrose, maltose, dextrans, or a combination thereof. In some embodiments, the excipient can include, but is not limited to, monosaccharides such as galactose, mannose, sorbose; disaccharides such as lactose, sucrose and trehalose and the like; polysaccharides such as starch, raffinose, dextran and the like; sugar alcohols (including erythritol, arabitol, xylitol, sorbitol, mannitol); cellulose-like polymers (including hydroxy cellulose, hydroxy propyl cellulose); insoluble additives (crystalline cellulose, chitosan, calcium carbonate, talc, titanium oxide) or silica (silicon oxide), and mixtures thereof. In one embodiment, the first and second excipients can be lactose monohydrate.

In some embodiments, the excipient comprises from about 0.01 to about 20 wt. % of the first layer, the second layer or the entire formulation. In some embodiments, the excipient comprises from about 0.1 to about 15 wt. %, from about 0.1 to about 10 wt. %, from about 0.1 to about 7 wt. %, from about 0.1 to about 5 wt. %, from about 0.1 to about 3 wt. %, from about 0.1 to about 1 wt. %, from about 1 to about 10 wt. %, from about 1 to about 7 wt. %, from about 1 to about 5 wt. %, from about 1 to about 3 wt. %, from about 3 to about 10 wt. %, from about 3 to about 7 wt. %, or from about 3 to about 5 wt. % of the first layer, the second layer or the entire formulation.

In some embodiments, the excipient comprises from about 0.1, 0.2, 0.3, 0.4, 0.5, 0.6, 0.7, 0.8, 0.9, 1, 2, 3, 4, 5, 6, 7, 8, 9, or 10 wt. % of the first layer, the second layer and/or the entire formulation. In one embodiment, the excipient is lactose monohydrate in an amount from about 18.0 mg to about 19.14 mg for a total pocket fill of from about 18.8 mg to about 19.5 mg.

In some embodiments, the excipient is a powder and has an average particle size of from about 10 to about 200 μm. In some embodiments, the excipient has an average particle size of from about 10 to about 150 μm, from about 10 to about 100 μm, from about 10 to about 75 μm, from about 10 to about 50 μm, from about 25 to about from about 150 μm, from about 25 to about 100 μm, from about 25 to about 75 μm, or from about 25 to about 50 μm. In some embodiments, the excipient has an average particle size of from about 10, 20, 30, 40, 50, 60, 70, 80, 90, 100, 110, 120, 130, 140, 150, 160, 170, 180, 190 to about 200 μm. The excipient can be a coarse powder or a fine powder or mixture thereof.

As with MGR002 dry powder formulation, the lactose mononhydrate utilized in the MGR003 dry powder formulation can be coarse or fine or a mixture thereof. In some embodiments, the lactose monohydrate is Lactohale 200 (LH200) having a particle size D₅₀ of from about 50 μm to about 100 μm or Lactohale 230 (LH230) having a particle size D₅₀ of less than about 10 μm.

Carboxamide hydrochloride was developed as a dry powder inhalation product (MGR002) and also in combination with MGR001, to provide a triple therapy dry powder inhalation product (MGR003). MGR002 drug product was developed to deliver a 128 μg fine particle mass (FPM) with a nominal dose of 441 μg per pocket of carboxamide hydrochloride. MGR003 was manufactured using a layer fill process, whereby carboxamide hydrochloride dry powder blend was filled first into the pocket of a CRC749 inhaler as the first layer and the fluticasone propionate and salmeterol xinafoate dry powder blend was filled as a second layer on top of the first layer within each individual pocket of a disc also present in the CRC749 inhaler. MGR003 product was developed to deliver a 128 μg FPM for the carboxamide hydrochloride component with a nominal dose of 250 μg/50 μg/441 μg of fluticasone propionate, salmeterol xinafoate and carboxamide hydrochloride per pocket. The fluticasone propionate and salmeterol xinafoate components of the MGR003 drug product are to meet the same performance targets as the MGR001 250/50 drug product.

In many aspects, the dry powder formulation containing 7% by weight of the carboxamide compound of formula I comprises, consists essentially of, or consists of a fine particle mass of 128 μg for a nominal dose of carboxamide compound of formula I of 441 μg. In certain aspects, the carboxamide compound of formula I has a mean fine particle fraction measured in percent total impact recovery from (TIR) from a next generation impactor (NGI) (FPF % TIR) of from about 31% to about 37%, values applicable to MGR003. Testing of the MGR003 product at 25° C. and 60% relative humidity (RH), 30° C. and 65% RH and 40° C. and 75% RH for about 6 months yielded fine particle mass (FPM) values from about 110 μg to about 160 μg. In other aspects, the dry powder formulation comprises a fine powder and the dose of the powder also known as the fine particle dose (FPD) is from about 20 μg to about 160 μg, and in yet other aspects, from about 161 μg to about 245 μg. Aerosolization performance data across the foregoing FPD range indicated fine particle fractions (FPFs) of from about 20% to about 40% or from about 25% to about 35%.

These and other aspects of the present application will be further appreciated upon consideration of the following examples, which are intended to illustrate certain particular embodiments of the application but are not intended to limit its scope, as defined by the claims

### EXAMPLES

### Example 1

In this example, a dry powder blend formulation of carboxamide hydrochloride and lactose monohydrate was obtained and utilized to provide the MGR002 and MGR003 products. To achieve a FPM of 128 μg of MGR002, it has been determined that a 7% w/w carboxamide hydrochloride dry powder blend was required. The formulation included coarse lactose only (LH200, D50 of 60 μm), with a 5% w/w API overage to account for losses during the blending process. This was based on a nominal fill weight of 6.3 mg with a fill weight overage of 5% to account for active pharmaceutical ingredient (API) losses during filling, which yielded a total pocket fill weight of 6.6 mg and a nominal dose of 441 μg, having a 29 fine particle fraction (FPF) % label claim (% LC).

The batch formulation and a unit formula per pocket for a carboxamide hydrochloride dry powder blend is illustrated below in Table 32.

TABLE 32

| | | | | Unit |
|---|---|---|---|---|
| | Quantity (g) | % | | formula/ |
| Ingredients | for 441 μg | w/w | | pocket |
| Carboxamide | 122.54¹ | 7.66 | | 0.482 mg |
| hydrochloride | | | | |
| Lactose | 1477.46 | 92.34 | | 5.818 mg |
| monohydrate LH200 | | | | |
| Total Batch Weight | 1600 | 100 | Total pocket | 6.300 mg² |
| | | | weight | |

¹A 5% w/w dispensing overage is added.
²A 5% w/w filling overage is added to give a total pocket fill weight of 6.6 mg.

Table 32 represents the batch formulation for the carboxamide hydrochloride dry powder blend component used in the MGR002 and MGR003 products. The 7% w/w carboxamide hydrochloride dry powder blend process was developed using conventional high shear blending, at 1.6 kg scale (4 L bowl) to 3.5 kg batch size (10 L bowl).

The dry powder blend was produced using a Diosna high shear blender. Both batch sizes use a 10 minute blending time, with 2 blend scrape downs during the blending step. Blend speeds were 600 rpm for 1.6 kg batch (4 L bowl) and 490 rpm for a 3.5 kg (10 L bowl). No cooling jacket was used and the blend yields expected were between 70% and 101% w/w. All development blends in this example were conducted at 45% RH±5% and 20° C.±2° C.

In the MGR003 product, the carboxamide hydrochloride dry powder blend MGR002 layer was filled first to the same fill weight targets as the MGR002 monotherapy product. Fluticasone propionate and salmeterol xinafoate dry powder blend of MGR001 was then filled to a target of 13 mg. This provided a total pocket fill weight for MGR003 of 19.6 mg. Filling station parameters illustrating the fill weight limits for carboxamide hydrochloride in MGR002 and MGR003 are shown in Table 33 for carboxamide hydrochloride dry powder blend for MGR002 and the first layer of MGR003 products.

Table 33 below illustrates the total fill weight for the MGR003 product.

TABLE 33

| Settings | 310 mm/s 2.0 G ≤ 80 Taps |
|---|---|
| Set up: | 19.40-19.80 |
| Mean Pocket Fill Weight Limits ± 1% (mg) | |
| During manufacture: | 19.21-19.99 |
| Mean Pocket Fill Weight Limits ± 2% (mg) | |
| Individual Pocket Weight Limits ± 6% (mg) | 18.43-20.77 |
| RSD (%) | ≤3.5 |

### Example 2

In this example, a formulation and blending process for the production of the MGR002 was developed. A MGR002 drug product was developed to deliver a 128 μg FPM with a nominal dose of 441 μg per pocket of carboxamide hydrochloride of formula II.

Micronized carboxamide hydrochloride at the predicted blend strength (7% w/w carboxamide) to achieve the desired FPM target are shown in Table 4. These batches contained either coarse Lactohale LH200 lactose having D₅₀ of 60 μm or coarse Lactohale LH200 lactose having D₅₀ of 80 μm. All batches were manufactured using the Diosna 4 L bowl which had the high shear blender and bowl size required to deliver a 1.6 kg of blend.

TABLE 34

| | Blend | | | |
|---|---|---|---|---|
| | strength | Speed | Time | Coarse |
| Batch | (% w/w) | (RPM) | (minutes) | lactose |
| 1 | 7.0 | 600.00 | 10.00 | 60 μm |
| 2 | 7.0 | 600.00 | 10.00 | 60 μm |
| 3 | 7.0 | 600.00 | 10.00 | 80 μm |
| 4 | 6.0 | 600.00 | 10.00 | 60 μm |
| 5 | 8.0 | 600.00 | 10.00 | 60 μm |

The blend yields of carboxamide compound of formula II and lactose monohydrate ranged from 74% to 92.%. 138 μg fine particle mass (FPM) was achieved using 7% weight by weight (w/w) blend strength of carboxamide hydrochloride with 60 μm of coarse lactose LH200. Batches of Table 34 were used to fill MGR002 and MGR003 products to support the drug product stability as discussed in Example 3 below.

### Example 3

In this example, MGR002 and MGR003 products were studied for stability using a 7% blend, 60 μm lactose monohydrate (Lactolase, LH200) and a 5% w/w API overage. A 5% API overage was recommended based on low potency of the blends resulting from losses to blender. The blend was obtained in a 4 L Diosna bowl by subjecting a 1.6 kg blend to 600 rpm and 10 minute blending process. All batches utilized in this study were filled at a set humidity range of 45% RH±5% and temperature of 20° C.±2° C. Fill weight targets used in this stability study are detailed in Tables 35 and 36 below.

TABLE 35

| | Target | Range |
|---|---|---|
| Set-up | (mg) | (mg) |
| Mean pocket fill weight (carboxamide | 7.00 ± 2.5% | 6.83-7.18 |
| hydrochloride blend, first layer) | | |
| Individual pocket weight (carboxamide | 7.00 ± 10.0% | 6.30-7.70 |
| hydrochloride blend, first layer) | | |
| % RSD (carboxamide hydrochloride | <3.50 | n/a |
| blend, first layer) | | |
| Mean pocket fill weight (first layer + | 20.00 ± 1.0% | 19.80-20.20 |
| fluticasone propionate and salmeterol | | |
| xinafoate dry powder blend, second | | |
| layer) | | |
| Individual pocket weight (first layer + | 20.00 ± 5.0% | 19.00-21.00 |
| second layer) | | |
| % RSD (first layer + second layer) | ≤3.50 | n/a |

RSD refers to relative standard deviation

TABLE 36

| | Target | Range |
|---|---|---|
| Manufacturing limits | (mg) | (mg) |
| Mean pocket fill weight (carboxamide | 7.00 ± 5.0% | 6.65-7.35 |
| hydrochloride blend, first layer) | | |
| Individual pocket weight (carboxamide | 7.00 ± 10.0% | 6.30-7.70 |
| hydrochloride blend, first layer) | | |
| % RSD (carboxamide hydrochloride | <3.50 | n/a |
| blend, first layer) | | |
| Mean pocket fill weight (first layer + | 20.00 ± 2.0% | 19.60-20.40 |
| fluticasone propionate and salmeterol | | |
| xinafoate dry powder blend, second | | |
| layer) | | |
| Individual pocket weight (first layer + | 20.00 ± 6.0% | 18.80-21.20 |
| second layer) | | |
| % RSD (first layer + second layer) | ≤3.50 | n/a |

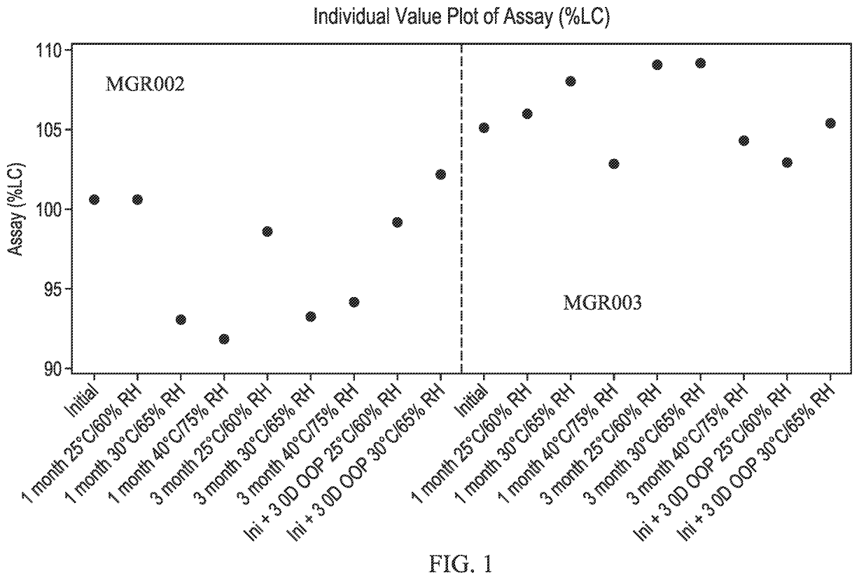

Table 35 sets up fill weight targets achieved for MGR003 and Table 36 illustrates manufacturing fill weight targets also achieved for MGR003. In Tables 35 and 36 above, MGR002 used in the first layer was 7% w/w, and contained in addition to carboxamide HCl also either 60 μm LH200 or 80 μm LH200 lactose monohydrate. For the second layer of fluticasone propionate and salmeterol xinafoate powder blend, MGR001 contained a 250 μg/50 μg blend of each active ingredient. By combining a first layer of MGR002 with a second layer of MGR001 in the pockets of a disc, a batch of MGR003 was obtained which contained 250 μg/50 μg/490 μg of fluticasone propionate and salmeterol xinafoate and carboxamide hydrochloride of formula II. Assay data for initial and 3 months stability were calculated based on the revised dose of 441 μg of carboxamide hydrochloride of formula II. FIG. 1 shows individual value plot of stability assays for batches of both MGR002 and MGR003 products taken at an initial time, 1 month, and 3 months at 25° C. and 60% RH, 30° C. and 65% RH, 40° C. and 75% RH. The data in FIG. 1 indicates that there was variability around the assay values across the stability conditions for both batches MGR002 and MGR003. This was thought to be caused by variability in filling, with regard to losses of the active pharmaceutical ingredient (API) during the filling process.

### Emitted Data

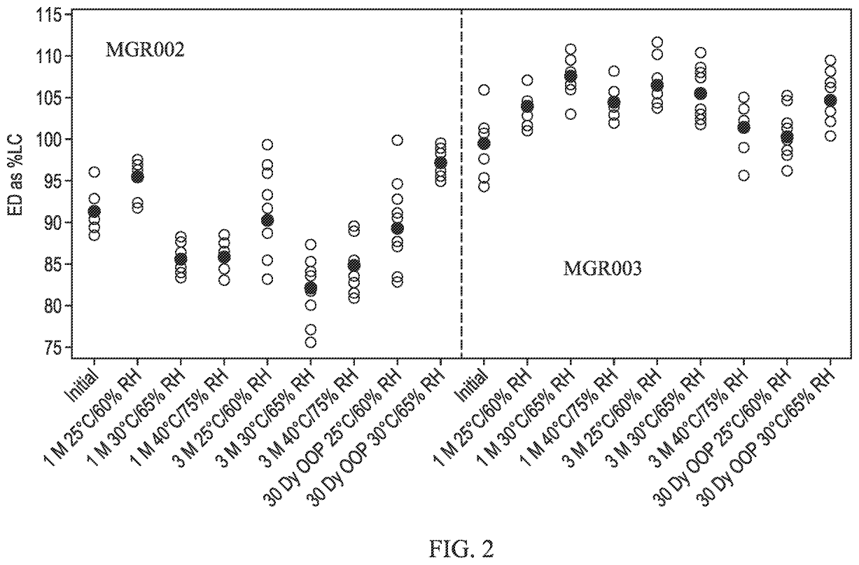

The emitted dose (ED) of carboxamide hydrochloride of formula II was calculated based on the revised nominal dose of 441 μg. FIG. 2 shows the emitted dose (% LC) of both MGR002 and MGR003 carboxamide hydrochloride layer batches. The emitted dose data followed the same trend as the assay data illustrated in FIG. 1, with MGR003 having a higher emitted dose on the average. Both batches, MGR002 and MGR003, met the drug product specification for assay (90% to 110% LC) and emitted dose (9 out of 10 are 75% to 125% of mean and all 10 within 50% to 150% of mean ED), respectively, at the initial time point and on the stability conditions reported.

### Aerosolization Performance

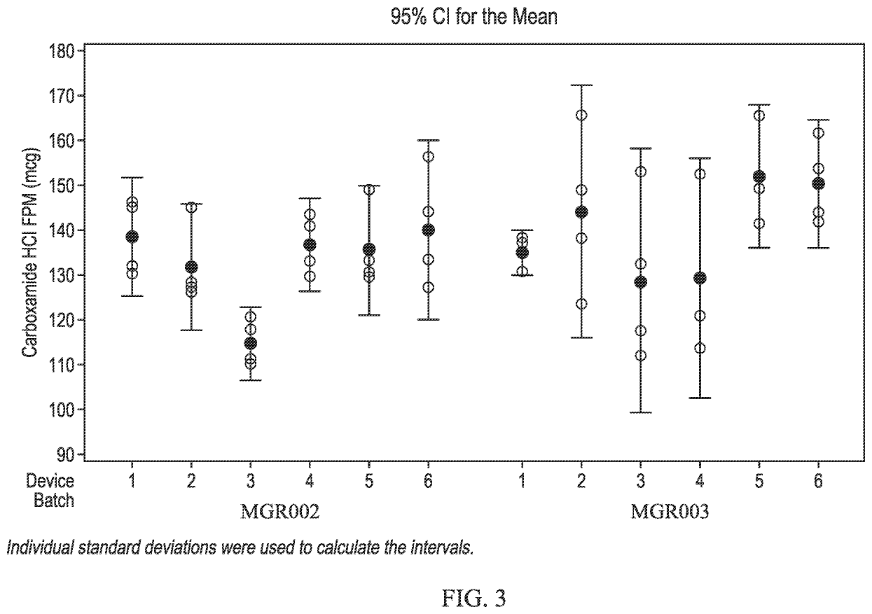

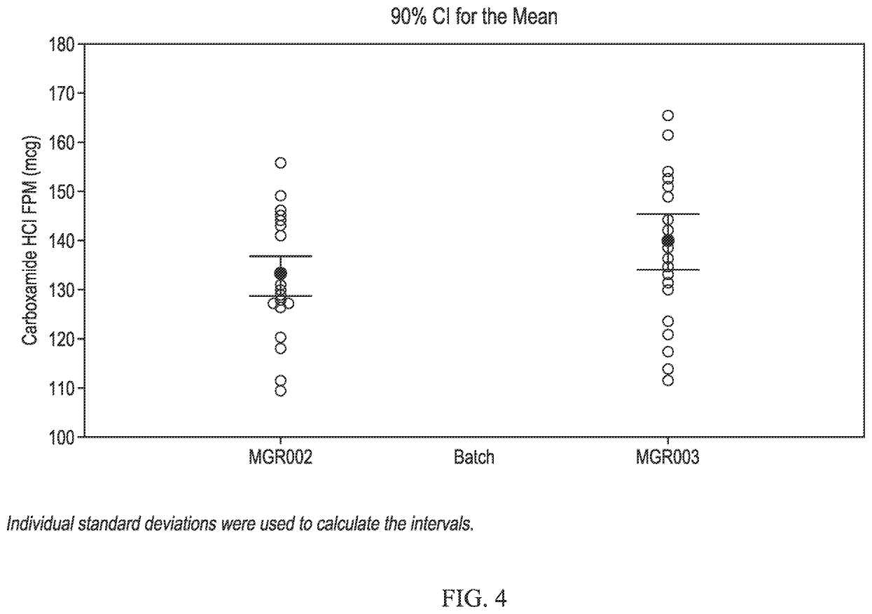

Aerosolization performance information was obtained for the same batches of MGR002 and MGR003 as were used in obtaining emitted data. FIG. 3 illustrates the aerosolization performance as measured by fine particle mass (FPM) obtained from 6 devices from each MGR002 and MGR003 product. FIG. 4 and Table 37 below present the results and equivalence criteria, respectively for carboxamide hydrochloride of formula II. The results in Table 37 are listed in terms of total impactor recovery (TIR) and fine particle mass (FPM) for MGR001, MGR002 and MGR003, carboxamide compound, salmeterol and fluticasone propionate.

TABLE 37

| | Ratio of | | | |
|---|---|---|---|---|
| | geometric means | | | |
| Carboxamide | (MGR002 divided | Lower | Upper | Pass/fail |
| Hydrochloride | by MGR003) | 90% CI | 90% CI | (0.90-1.11) |
| TIR | 0.97 | 0.95 | 0.99 | Pass |
| FPM | 0.95 | 0.91 | 1.00 | Pass |

| | Ratio of | | | |
|---|---|---|---|---|
| | geometric means | | | |
| | (MGR001 divided | Lower | Upper | Pass/fail |
| | by MGR003) | 90% CI | 90% CI | (0.90-1.11) |
| Salmeterol | | | | |
| TIR | 0.97 | 0.96 | 0.98 | Pass |
| FPM | 1.01 | 0.97 | 1.05 | Pass |
| Fluticasone | | | | |
| propionate | | | | |
| TIR | 0.96 | 0.93 | 0.98 | Pass |
| FPM | 0.99 | 0.95 | 1.03 | Pass |

The mean particle mass (FPM) and mean total impactor recovery (TIR) from a next generation impactor (NGI) for each batch were equivalent, based on the fact that the upper/lower 90% confidence interval of the ratio of the geometric mean, and fell between 0.90 and 1.11. Table 37 also shows the results and equivalence criteria for salmeterol xinafoate and fluticasone propionate for batches MGR001 and MGR003. The fluticasone propionate and salmeterol xinafoate components of the MGR003 drug product also met the same performance targets as the MGR001 250/50 drug product.

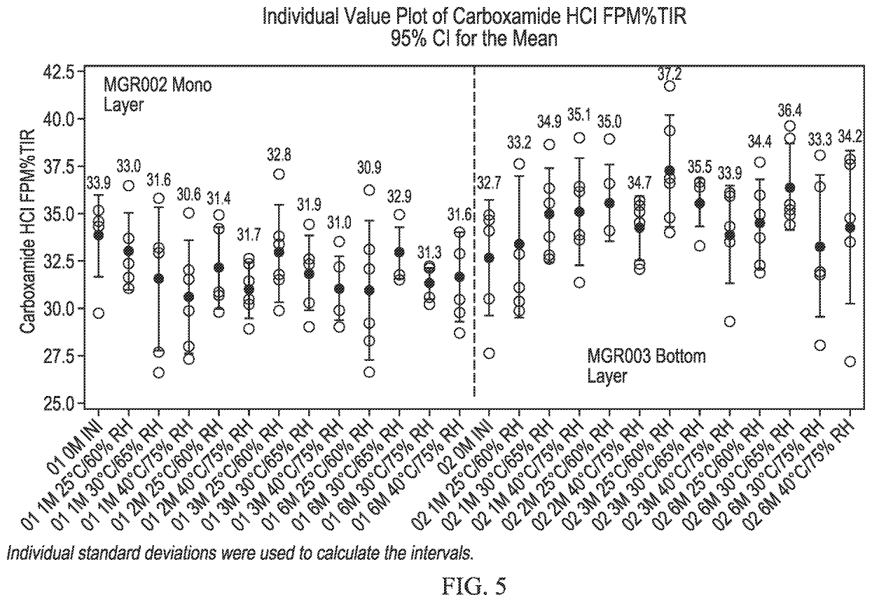

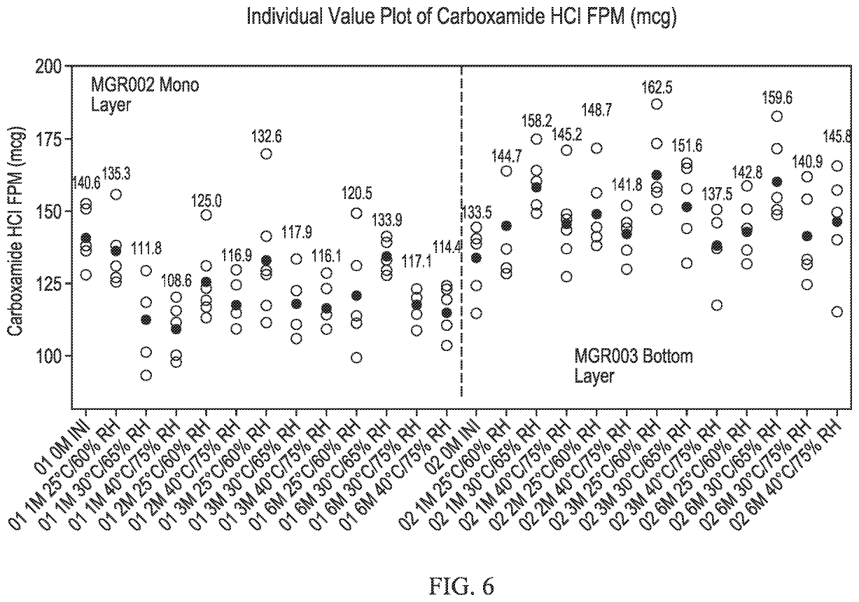

Testing of both MGR002 and MGR003 products between an initial time and 6 months at 25° C./60% RH, 30° C./65% RH, and 40° C./75% RH yielded a mean FPF % TIR values in a range from about 31 to about 37 mean data labels as shown in FIG. 5. FPM values between from about 110 μg to about 160 μg mean data labels as shown in FIG. 6 were obtained without any observable trends on stability. Both MGR002 and MGR003 equivalence study demonstrated that the FPM of carboxamide compound is equivalent for both products, using the stability batches of carboxamide compound of formula I and also MGR001 is equivalent to MGR003 for salmeterol (as xinafoate) and fluticasone propionate. This example has shown a robust and stable aerosolization performance, up to 3 months at 25° C./60% RH and 40° C./75% RH, when filled into a CRC749 device and foil pouch was provided as illustrated in FIGS. 5 and 6.

### Stability Studies

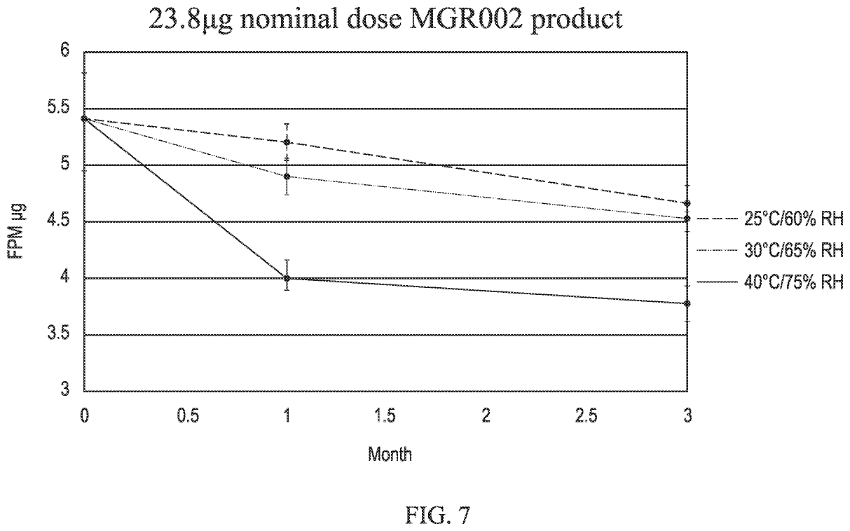

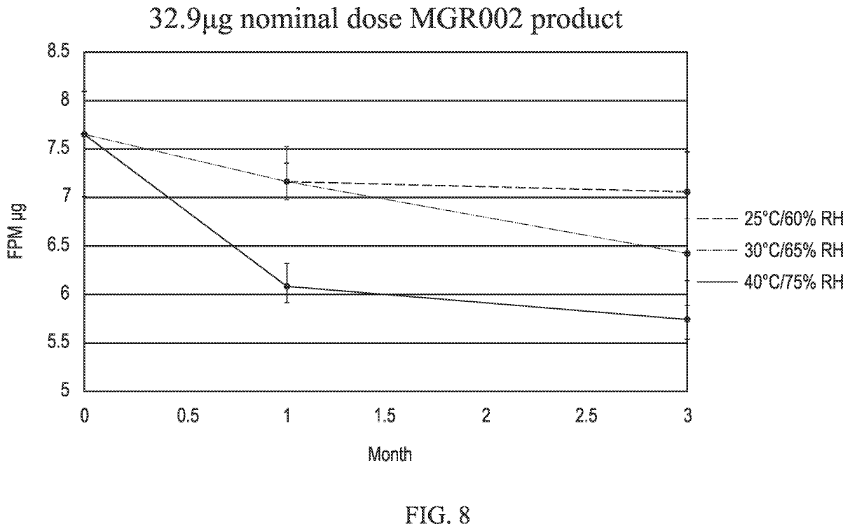

Stability studies were performed on a nominal dose of a 23.8 μg MGR002 product and a nominal dose of a 32.9 μg MGR002 product. The stability plots for both product strengths are illustrated in FIGS. 7 and 8. The initial time point for the 23.8 μg product produced an FPM value of 5.4 μg. Results showed a stability drop to 4.7 μg after three months at 25° C./60% RH. After three months at an accelerated storage condition of 40° C./75% RH, the FPM dropped to 3.8 μg, as shown in FIG. 7.

The initial time point for the 32.9 μg product produced an FPM value of 7.7 μg. Results showed a stability drop to 7.1 μg after three months at 25° C./60% RH and 5.7 μg at 40° C./75% RH, as shown in FIG. 8.

Stability studies were performed on a batch of MGR002 (Batch No. 00900931). The storage conditions, maximum storage time and batch number are shown below in Table 38.

TABLE 38

| Storage Condition | Maximum Storage Time | Batch |
|---|---|---|
| 25° C./60% RH | 12 months | 00900931 |
| 30° C./65% RH | 12 months | |
| 40° C./75% RH | 6 months | |
| Photostability | 1.2 × 10⁶ lux hours & | |
| | UV 200 watt hours/m² | |

The data indicated that MGR002 is chemically stable over 12 months at the storage conditions of 25° C./60% RH and 30° C./65% RH, and over 6 months at the accelerated condition of 40° C. 175% RH. No significant changes have been observed over these time periods. MGR002 has also been found to be stable after light exposure during photostability testing.

The stability protocol is shown below in Table 39.

TABLE 39

| | Storage Time | | | | | | | |
|---|---|---|---|---|---|---|---|---|
| | | 6 | 3 | 6 | 12 | 18 | 24 | 36 |
| Storage Condition | Initial | weeks | months | months | months | months | months | months |
| 25° C./60% RH | X | W | W | X | X | W | X | X |
| 30° C./65% RH | | W | W | X | X | W | X | X |
| 40° C./75% RH | | W | W | X | NS | NS | NS | NS |

W = Appearance, polymorphism., particle size, organic impurities, assay, water content, residual solvents and volatile process related impurities.
X = Appearance, polymorphism, particle size, organic impurities, assay, water content, microbial enumeration, residual solvents and volatile process related impurities.
NS = Not scheduled

In summary, up to 12 months of stability data was reported for Batch No. 00900931 of MGR002. No significant changes were observed in the data reported at any storage conditions or time point. The stability data presented support the storage condition of not more than 25° C. (with transient spikes up to 40° C. permitted for periods of not more than 24 hours), when packed in double polyethylene bags inside a heat sealed foil pouch.

Stability data was performed on a batch of MGR003 (Batch No. 800-009-DE-02). The batch was packaged in foil laminate overwrap placed within a cardboard carton. Stability testing was conducted at 25° C./60% RH, 30° C./65% RH, 30° C./75% RH and 40° C./75% RH in a vertical orientation, as shown in Table 40 below.

TABLE 40

| Storage Conditions | Initial | 1M | 2 M | 3 M | 6 M | 12 M |
|---|---|---|---|---|---|---|
| Initial | X | | | | | |
| 25° C./60% RH | | X | Y | X | X | X |
| 30° C./65% RH | | X | | X | X | X |
| 30° C./75% RH | | | | | Z | Z |
| 40° C./75% RH | | X | Y | X | X | |

W = Appearance, polymorphism, particle size, organic impurities, assay, water content, residual solvents and volatile process related impurities.
X = Appearance, polymorphism, particle size, organic impurities, assay, water content, microbial enumeration, residual solvents and volatile process related impurities.
NS = Not scheduled

In-use (out of pouch) stability data was also generated on the same batch after removal from the foil laminate overwrap, then returned to the carton and storing at 25° C./60% RH and 30° C./65% RH to support an in-use period as detailed in Table 41 below.

TABLE 41

| | Initial + | Initial + 30 | 4 M + 45 | 4 M + 60 | 10 M + 45 | 10 M + 60 |
|---|---|---|---|---|---|---|
| Storage Conditions | 15 days | days | days | days | days | days |
| 25° C./60% RH | X | Y | X | Y | X | Y |
| 30° C./65% RH | X | Y | X | Y | X | Y |

X = indicates that the following tests will be performed: degradation products and aerodynamic particle size distribution
Y = indicates that the following tests will be performed: powder description, device description, assay, degradation products, uniformity of delivered dose, aerodynamic particle size distribution and water.

The data indicated that the MGR003 inhalation powder is stable over 6 months at the storage conditions of 25° C./60% RH, 30° C./65% RH, 30° C./75% RH. No significant changes have been observed up to 6 months for any tests at long term storage conditions. There was a significant change observed for degradation products at 40° C./75% RH through 6 months.

It will be apparent to those of ordinary skill in the art that various modifications and variations can be made to various embodiments described herein without departing from the spirit or scope of the teachings herein. Thus, it is intended that various embodiments cover other modifications and variations of various embodiments within the scope of the present teachings.

1. A powder formulation for use in a dry powder inhaler, the powder formulation comprising from about 0.01% by weight to about 90% by weight of a carboxamide compound of formula I 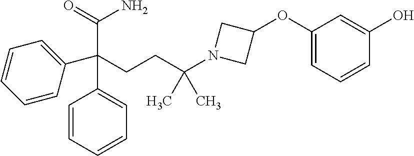 [SMILES: CC(C)(CCC(C(N)=O)(c1ccccc1)c1ccccc1)N1CC(Oc2cccc(O)c2)C1]
or a pharmaceutically acceptable salt thereof and an excipient.

2. The powder of claim 1, wherein the carboxamide compound is a compound of formula II

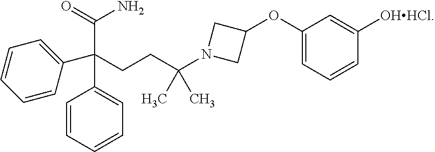

3. The powder of claim 1, wherein the carboxamide compound is 5-[3-(3-Hydroxyphenoxy)azetidin-1-yl]-5-methyl-2,2-diphenylhexanamide hydrochloride.

4. The powder of claim 1, wherein the powder is dry and comprises: (i) from about 0.25% by weight to about 10% by weight of the carboxamide compound of formula I; or (ii) from about 0.34% by weight to about 6.00% by weight of the carboxamide compound of formula I.

5. The powder of claim 1, wherein the inhaler comprises a plurality of pockets, each pocket configured to contain the carboxamide compound at a nominal fill weight of from about 6.3 mg to about 7.0 mg.

6. The powder of claim 5, wherein a nominal dose of carboxamide compound of formula 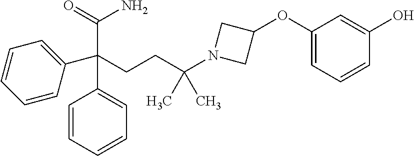 [SMILES: CC(C)(CCC(C(N)=O)(c1ccccc1)c1ccccc1)N1CC(Oc2cccc(O)c2)C1]
per nominal pocket fill comprises: (i) from about 0.018 mg to about 0.7 mg for a total nominal fill weight in the pocket of 7 mg; or (ii) from about 0.024 mg to about 0.42 mg for a total nominal fill weight in the pocket of 7 mg; or (iii) about 0.441 mg for a total nominal fill weight in the pocket of about 7 mg.

7. The powder of claim 5, wherein a nominal dose of carboxamide compound of formula II 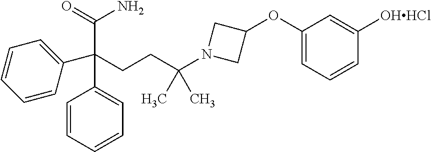
per nominal pocket fill comprises: (i) from about 0.019 mg to about 0.757 mg for a total nominal fill weight in the pocket of about 7 mg; or (ii) about 0.026 mg to about 0.454 mg nominal fill weight in the pocket of about 7 mg; or (iii) about 0.477 mg nominal fill weight in the pocket of about 7 mg.

8. The powder of claim 1, wherein the excipient comprises: (i) monosaccharides, disaccharides, oligosaccharides or polysaccharides, or a combination thereof; (ii) glucose, arabinose, lactose, sucrose, maltose, dextrans, or lactose, or a combination thereof; (iii) lactose monohydrate; (iv) phosphatidylcholine, 1-leucine, mannitol, or magnesium stearate; or (v) menthol, levomenthol, saccharin, or saccharin sodium, or a combination thereof.

9. The powder of claim 8, wherein the lactose monohydrate is in the powder in: (i) an amount from about 5.86 mg to about 6.98 mg; or (ii) an amount from about 5.86 mg to about 6.98 mg.

10. The powder of claim 8, wherein the lactose monohydrate comprises particles having a mass-median-diameter D50 from about 60 μm to about 80 μm.

11. The powder of claim 1, wherein the excipient comprises from about 10% by weight to about 99.5% by weight of the formulation.

12. The powder of claim 4, wherein: (i) the dry powder comprises fine particles and coarse particles and the ratio between the fraction of fine particles and the fraction of coarse particles is between about 0.25 and about 100; or (ii) the carboxamide compound of formula I or formula II has a D90 particle size from about 5 μm to about 10 μm.

13. The powder of claim 4, wherein about 7% by weight of the carboxamide compound of formula I comprises a fine particle mass of about 128 μg for a nominal dose of carboxamide compound of about 441 μg.

14. The powder of claim 4, wherein: (i) the carboxamide compound of formula I has a mean fine particle fraction of from about 31% to about 37% total impactor recovery; or (ii) the carboxamide compound of formula I has a particle mass from about 110 μg to about 160 μg.

15. The powder of claim 4, wherein the powder is a fine powder having a fine particle dose of from about 20 μg to about 160 μg; or from about 161 μg to about 245 μg.

16. A powder formulation for use in a dry powder inhaler, the powder comprising a carboxamide compound of formula I 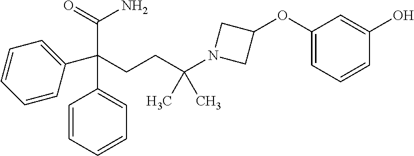 [SMILES: CC(C)(CCC(C(N)=O)(c1ccccc1)c1ccccc1)N1CC(Oc2cccc(O)c2)C1]
or a pharmaceutically acceptable salt thereof, a pharmaceutically acceptable excipient, and a long acting β2 adrenoceptor agonist, and optionally a corticosteroid.

17. The powder formulation of claim 16, wherein the carboxamide compound is 5-[3-(3-Hydroxyphenoxy) azetidin-1-yl]-5-methyl-2, 2-diphenylhexanamide hydrochloride of formula II 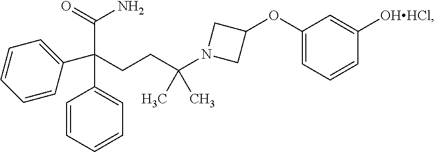
the β2 adrenoceptor agonist comprises salmeterol xinafoate and the corticosteroid comprises fluticasone propionate.

18. The powder formulation of claim 16, wherein the powder contains from about 0.8% by weight to about 10% by weight of the carboxamide compound of formula I.

19. The powder formulation of claim 16, wherein the excipient comprises: (i) monosaccharides, disaccharides, oligosaccharides or polysaccharides, or a combination thereof; (ii) glucose, arabinose, lactose, sucrose, maltose, dextrans, or lactose, or a combination thereof; (iii) lactose monohydrate; (iv) phosphatidylcholine, 1-leucine, mannitol, or magnesium stearate; or (v) menthol, levomenthol, saccharin, or saccharin sodium, or a combination thereof.

20. The powder formulation of claim 19, wherein the dry powder inhaler comprises a plurality of pockets, each pocket configured to comprise at least two blend layers, wherein the at least two blend layers comprise:
(i) a first blend containing from about 0.8% to about 10% by weight of the carboxamide compound of formula I filled to a nominal weight of about 7 mg in the pocket and a second blend containing about 2% by weight of fluticasone propionate and about 0.4% by weight salmeterol filled to a nominal 12.5 mg in the pocket, both blends filled to a total nominal pocket fill weight of from about 18.8 mg to about 19.5 mg; or
(ii) a first blend containing from about 0.056 mg to about 0.441 mg of the carboxamide compound of formula I and from about 5.86 mg to about 6.94 mg of lactose monohydrate filled to a nominal fill weight of from about 6.3 mg to about 7.0 mg, and a second blend containing from about 0.05 mg salmeterol, about 0.250 mg of fluticasone propionate and about 12.2 mg lactose monohydrate, both blends filled to a total pocket fill weight of from about 18.8 mg to about 19.5 mg for a total pocket fill weight of from about 18.8 mg to about 19.5 mg; or
(iii) a first blend containing from about 0.87 mg to about 10.82 mg of the carboxamide compound of formula II filled to a nominal 7 mg of powder added to the pocket, and a second blend containing from about 2.00 mg of fluticasone propionate, from about 0.58 mg of salmeterol xinafoate, both blends filled to a total nominal fill weight of from about 18.8 mg to about 19.5 mg; or
(iv) a first blend from about 0.061 mg to about 0.477 mg of the carboxamide compound of formula II and from about 5.823 mg to about 6.939 mg of lactose mononhydrate for a nominal fill weight of about 7.0 mg, and a second blend from about 0.250 mg of fluticasone propionate and about 0.073 mg of salmeterol xinafoate and about 12.177 mg lactose monohydrate for a nominal fill weight of about 12.5 mg, both blends filled to a total nominal fill weight of from about 18.8 to about 19.5.

21.-30. (canceled)
